(19) United States
(12) Patent Application Publication
(10) Pub. No.: US 2012/0323183 A1
(43) Pub. Date: Dec. 20, 2012

(54) SELF-INJECTION DEVICE

(76) Inventors: Bart Peterson, Farmington, UT (US); Ralph Sonderegger, Farmington, UT (US); Christopher Cindrich, Draper, UT (US)

(21) Appl. No.: 13/516,122

(22) PCT Filed: Dec. 16, 2009
(86) PCT No.: PCT/US09/06577

§ 371 Date: Aug. 27, 2012

(51) Int. Cl. A61M 5/32; A61M 5/31

(52) U.S. Cl. 604/180; 604/195

(57) ABSTRACT

A drug delivery device (500), including a body (504) having a needle opening (508) and a reservoir (164, 176) disposed therein for containing a medicament, and an injection needle (152) for penetrating the skin of a patient, the needle (152) providing a path for the medicament between the reservoir (164, 176) and the patient, and selectively protruding from the body (504) through the needle opening (508). The device (500) also includes safety means (576, 548, 552, 532, 512) for automatically retracting the needle (152) within the body (504) and covering the needle opening (508) upon removal of the device (500) from the patient.

### FIELD OF THE INVENTION

The present invention relates generally to a substance delivery device having improved patient convenience and ease of use, and improved safety mechanisms. The present invention also relates generally to a patch-like, self-contained substance infusion or self-injection device that can be used to deliver a variety of substances or medications to a patient. More specifically, the present invention relates to safety mechanisms for patch-like infusion or self-injection devices.

### BACKGROUND OF THE INVENTION

A large number of people, such as those suffering from conditions such as diabetes, use some form of infusion therapy, such as daily insulin infusions, to maintain close control of their glucose levels. Currently, in the insulin infusion treatment example, there are two principal modes of daily insulin therapy. The first mode includes syringes and insulin pens. These devices are simple to use and are relatively low in cost, but they require a needle stick at each injection typically three to four times per day. The second mode includes infusion pump therapy, which entails the purchase of an expensive pump that lasts for about three years. The high cost (roughly 8 to 10 times the daily cost of syringe therapy) and limited lifetime of the pump are high barriers to this type of therapy. Insulin pumps also represent relatively old technology and are cumbersome to use. From a lifestyle standpoint, moreover, the tubing (known as the “infusion set”) that links the pump with the delivery site on the patient's abdomen is very inconvenient and the pumps are relatively heavy, making carrying the pump a burden. From a patient perspective, however, the overwhelming majority of patients who have used pumps prefer to remain with pumps for the rest of their lives. This is because infusion pumps, although more complex than syringes and pens, offer the advantages of continuous infusion of insulin, precision dosing and programmable delivery schedules. This results in closer glucose control and an improved feeling of wellness.

Interest in better therapy is on the rise, accounting for the observed growth in pump therapy and increased number of daily injections. In this and similar infusion examples, what is needed to fully meet this increased interest is a form of insulin delivery or infusion that combines the best features of daily injection therapy (low cost and ease of use) with those of the insulin pump (continuous infusion and precision dosing) and that also avoids the disadvantages of each.

Several attempts have been made to provide ambulatory or “wearable” drug infusion devices that are low in cost and convenient to use. Some of these devices are intended to be partially or entirely disposable. In theory, devices of this type can provide many of the advantages of an infusion pump without the attendant cost and inconvenience. Unfortunately, however, many of these devices suffer from disadvantages including patient discomfort (due to the gauge and/or length of injection needle used), compatibility and interaction between the substance being delivered and the materials used in the construction of the infusion device, and possible malfunctioning if not properly activated by the patient (for example, “wet” injections resulting from premature activation of the device). Difficulties in manufacturing and in controlling needle penetration depth have also been encountered, particularly when short and/or fine-gauge injection needles are used. The possibility of needle-stick injuries to those who come into contact with the used device has also been problematic.

Accordingly, a need exists for an alternative to current infusion devices, such as infusion pumps for insulin, that further provides simplicity in manufacture and use improvements for insulin and non-insulin applications.

### SUMMARY OF THE INVENTION

An aspect of the present invention is to provide a patch-like infusion or self-injection device that can be conveniently worn against the skin while providing infusion of a desired substance, and providing minimal discomfort by using one or more microneedles. An additional aspect of the present invention is to provide a safety mechanism for such an infusion or self-injection device.

The foregoing and/or other aspects of the present invention are achieved by providing a drug delivery device, including a body having a needle opening and a reservoir disposed therein for containing a medicament, and an injection needle for penetrating the skin of a patient, the needle providing a path for the medicament between the reservoir and the patient, and selectively protruding from the body through the needle opening. The device also includes safety means for automatically retracting the needle within the body and covering the needle opening upon removal of the device from the patient.

The foregoing and/or other aspects of the present invention are also achieved by providing a safety mechanism for a drug delivery device including a body having a needle opening, a reservoir disposed in the body for containing a medicament, and an injection needle for penetrating the skin of a patient, the needle providing a path for the medicament between the reservoir and the patient, and selectively protruding from the body through the needle opening. The safety mechanism includes a safety member movable within the body from a pre-deployed position to a covered position covering the needle opening, a biasing mechanism biasing the safety member toward the covered position upon activation of the drug delivery device, and a blocker movably connected to the body for selectively preventing movement of the safety member toward the covered position prior to deployment of the safety mechanism. Movement of the safety member to the covered position retracts the needle within the body.

The foregoing and/or other aspects of the present invention are also achieved by providing a drug delivery device including a body having a surface placeable on the skin of a patient, the body having a needle opening and a reservoir disposed therein for containing a medicament, and an injection needle for penetrating the skin of a patient, the needle providing a path for the medicament between the reservoir and the patient, and being selectively movable between a first position, in which at least a part of the needle is contained within the body, and a second position, in which at least a part of the needle protrudes from the body through the needle opening. The drug delivery device also includes a safety mechanism pivotably connected to the body and being selectively pivotable between a retracted position and a deployed position shielding the injection needle, the safety mechanism having a safety surface placeable on the skin of the patient, the safety surface having an adhesive disposed thereon for adhering the safety mechanism to the skin of the patient and thereby automatically pivoting the safety mechanism to the second position upon removal of the drug delivery device from the skin of the patient.

Additional and/or other aspects and advantages of the present invention will be set forth in part in the description that follows and, in part, will be apparent from the description, or may be learned by practice of the invention.

### BRIEF DESCRIPTION OF THE DRAWINGS

The above and/or other aspects and advantages of embodiments of the invention will be more readily appreciated from the following detailed description, taken in conjunction with the accompanying drawings, in which:

FIG. 1 illustrates a perspective view of an embodiment of a patch-like infusion or self-injection device in a pre-activated state prior to activation;

FIG. 2 illustrates a partially exploded view of the infusion device of FIG. 1 in the pre-activated state;

FIG. 3 illustrates a partially exploded view of the infusion device of FIG. 1 in the pre-activated state with an activator button rotated away to reveal more detail;

FIG. 4 illustrates a more fully exploded view of the infusion device of FIG. 1 in the pre-activated state;

FIG. 5 illustrates a cross-sectional view of the infusion device of FIG. 1 in the pre-activated state;

FIG. 6 illustrates a cross-sectional view of the infusion device of FIG. 1 in the pre-activated state with the activator button rotated away;

FIG. 7 illustrates a partially exploded view of the infusion device of FIG. 1 during installation of a safety mechanism;

FIG. 8 illustrates a partially exploded view of the infusion device of FIG. 1 subsequent to activation;

FIG. 9 illustrates a more fully exploded view of the infusion device of FIG. 1 subsequent to activation;

FIG. 10 illustrates a cross-sectional view of the infusion device of FIG. 1 subsequent to activation;

FIG. 11 illustrates a partially exploded view of the infusion device of FIG. 1 subsequent to deployment of the safety mechanism;

FIG. 12 illustrates a cross-sectional view of the infusion device of FIG. 1 subsequent to deployment of the safety mechanism;

FIG. 13 illustrates a bottom surface of the safety mechanism;

FIG. 14 further illustrates the structure of the safety mechanism;

FIGS. 15A-15D illustrate an end-of-dose indicator and the operation thereof in the infusion device of FIG. 1;

FIG. 16 illustrates an embodiment of an infusion device with an injection port;

FIG. 17 illustrates an embodiment of an infusion device with another embodiment of a safety mechanism;

FIG. 18 illustrates a blocker of the infusion device of FIG. 17;

FIG. 19 illustrates a partially exploded view of the infusion device of FIG. 17 in the pre-activated state;

FIG. 20 illustrates an activator button of the infusion device of FIG. 17;

FIG. 21 illustrates a cross-sectional view of the infusion device of FIG. 17 in a pre-activated state with the blocker of FIG. 18;

FIG. 22 illustrates a partial cross-sectional view of the infusion device of FIG. 17 in a pre-activated state with another embodiment of a blocker;

FIG. 23 illustrates a more fully exploded view of the infusion device of FIG. 17 in the pre-activated state;

FIG. 24 illustrates a cross-sectional view of the infusion device of FIG. 17 in an activated state;

FIG. 25 illustrates a partially exploded view of the infusion device of FIG. 17 in the activated state;

FIG. 26 illustrates interaction between a safety member and a needle manifold when the safety mechanism of the infusion device of FIG. 17 is deployed; and

FIG. 27 illustrates the safety member in a covered position.

### DETAILED DESCRIPTION OF EXEMPLARY EMBODIMENTS

Reference will now be made in detail to embodiments of the present invention, examples of which are illustrated in the accompanying drawings, wherein like reference numerals refer to the like elements throughout. The embodiments described exemplify the present invention by referring to the drawings.

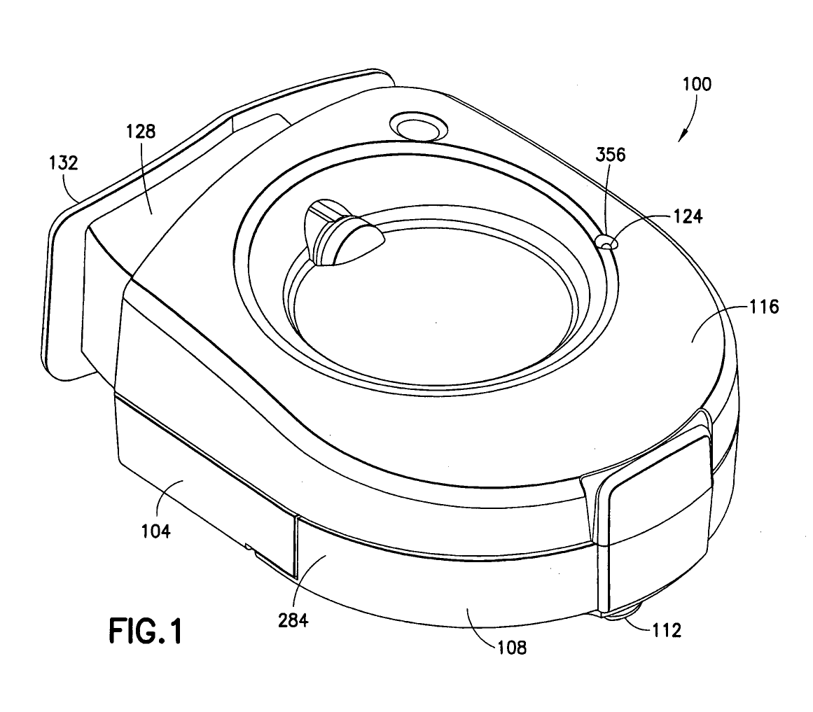

The embodiments of the present invention described below can be used as a convenient, patch-like infusion or self-injection device 100 to deliver a pre-measured dose of a substance, such as a liquid drug or medication, to a patient over a period of time or all at once. The device is preferably provided to the end user in a pre-filled condition, that is, with the drug or medication already in the device reservoir. Though the patch-like infusion or self-injection device 100 (shown, for example, in FIG. 1) described herein can be employed by a patient and/or a caregiver, for convenience, a user of the device is hereinafter referred to as a “patient.” Additionally, for convenience, terms such as “vertical” and “horizontal” and “top” and “bottom” are employed to represent relative directions with respect to an infusion device 100 disposed on a horizontal surface. It will be understood, however, that the infusion device 100 is not limited to such an orientation, and that the infusion device 100 may be employed in any orientation. Further, the alternative use of the terms “infusion device” and “self-injection device” to describe devices embodying the present invention is not intended in a limiting sense. Infusion devices that do not have a self-injection capability are within the scope of the present invention, as are self-injection devices that do not carry out continuous infusion. For convenience, but not by way of limitation, the term “infusion device” is used in the description that follows.

The patch-like infusion device 100 of FIG. 1 is self-contained and is attached to the skin surface of the patient by adhesive disposed on a bottom of the infusion device 100 (as will be described in greater detail below). Once properly positioned and activated by the patient, the pressure of a released spring on a flexible reservoir within the device can be used to empty the contents of the reservoir through one or more patient needles (for example, microneedles) via a needle manifold. The substance within the reservoir is then delivered through the skin of the patient by the microneedles, which are driven into the skin. It will be understood that other embodiments are possible in which the spring is replaced with a different type of stored energy device, which may be mechanical, electrical and/or chemical in nature.

As will be appreciated by one skilled in the art, there are numerous ways of constructing and using the patch-like infusion device 100 disclosed herein. Although reference will be made to the embodiments depicted in the drawings and the following descriptions, the embodiments disclosed herein are not meant to be exhaustive of the various alternative designs and embodiments that are encompassed by the disclosed invention. In each disclosed embodiment, the device is referred to as an infusion device, but the device may also inject substances at a much faster (bolus) rate than is commonly accomplished by typical infusion devices. For example, the contents can be delivered in a period as short as several seconds or as long as several days.

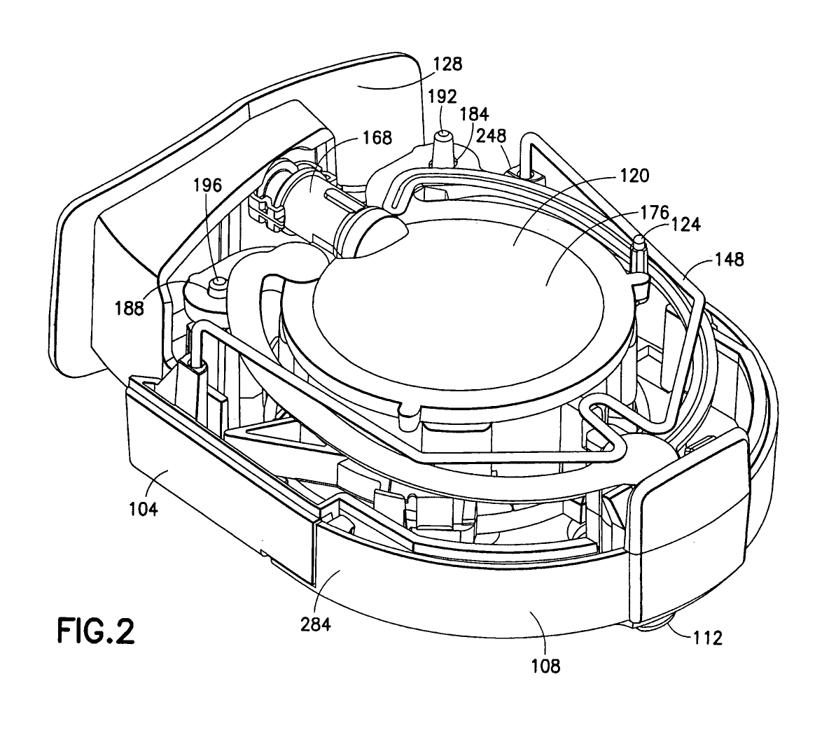

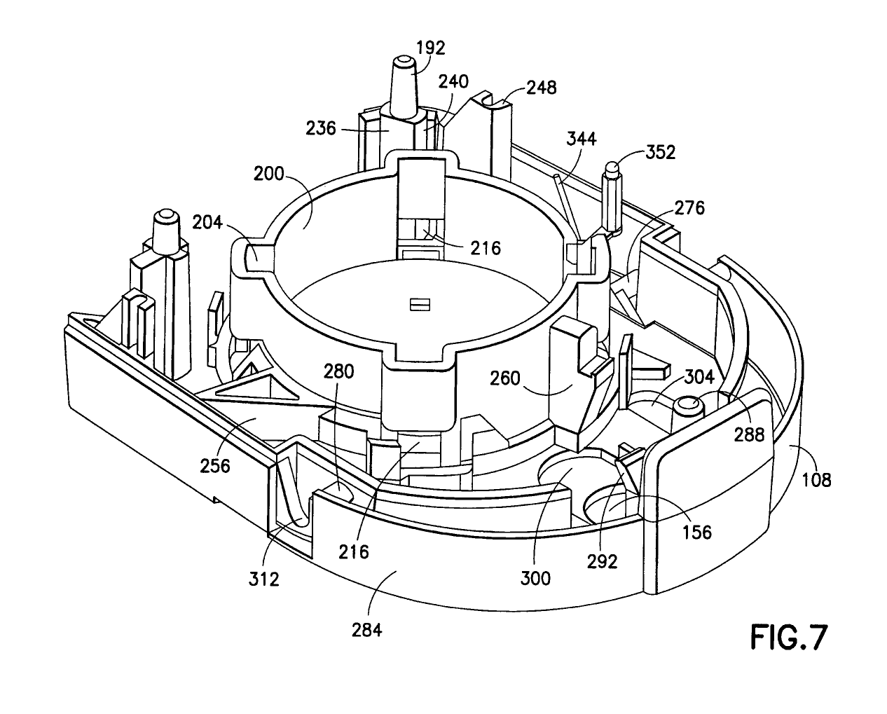

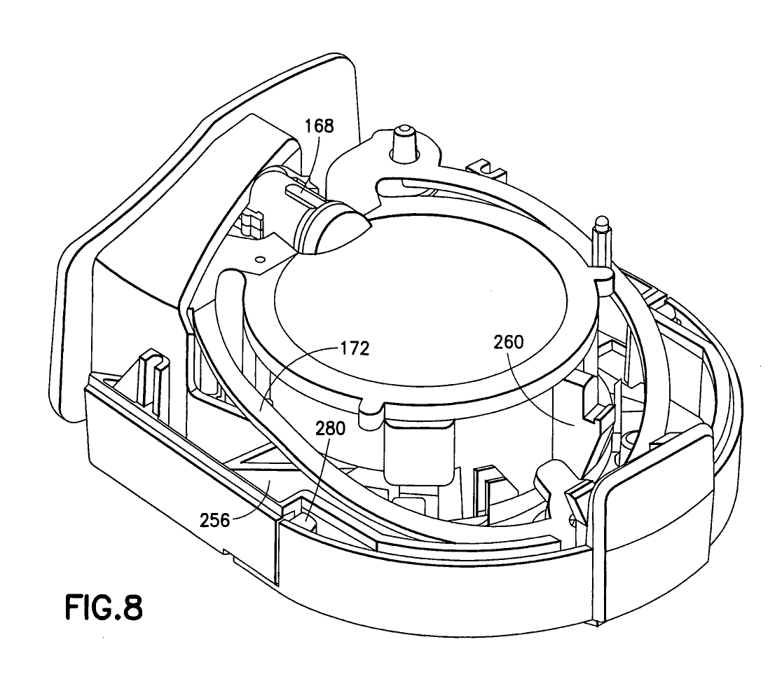

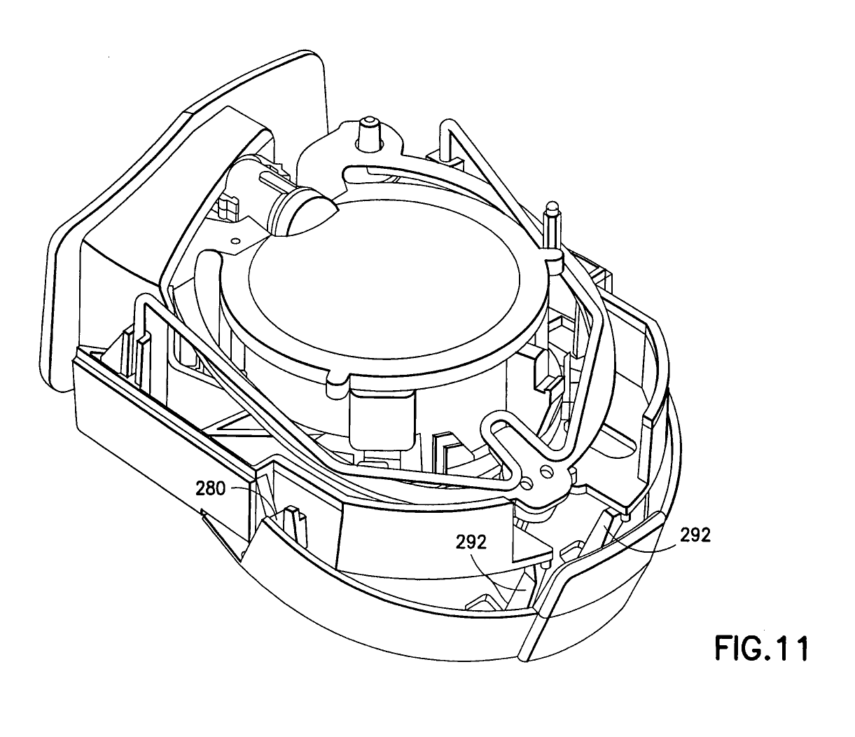

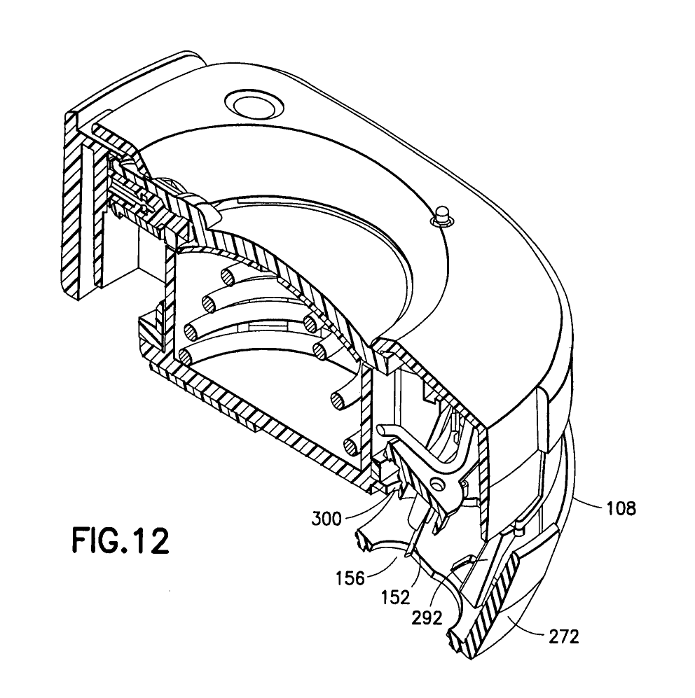

In an embodiment of the device shown in FIGS. 1 through 12, a push-button design of the patch-like infusion device 100 is shown wherein the activation and energizing of the device is accomplished in a single multi-function/step process. FIG. 1 illustrates an assembled embodiment of the infusion device 100 in a pre-activated state. FIGS. 2-6 illustrate partially exploded and cross-sectional views of the infusion device 100 in the pre-activated state, FIG. 7 illustrates a partially exploded view of the infusion device 100 during installation of a safety mechanism, FIGS. 8-10 illustrate exploded and cross-sectional views of the infusion device 100 subsequent to activation, and FIGS. 11 and 12 illustrate exploded and cross-sectional views of the infusion device 100 subsequent to deployment of the safety mechanism. The infusion device 100 is configured to operate between the pre-activated state (shown, for example, in FIGS. 1, 2, and 5), an activated or fired state (shown, for example, in FIGS. 8-10), and a retracted or safe state (shown, for example, in FIGS. 11 and 12).

As shown in FIG. 1, an embodiment of the patch-like infusion device 100 includes a bottom enclosure 104, a safety mechanism 108, a flexible needle cover 112, a top enclosure 116, a reservoir subassembly 120, an end-of-dose indicator (EDI) 124, and an activator button 128, which includes a patient interface surface 132. Additionally, as shown in FIGS. 2-6, the infusion device 100 also includes a rotor or activation ring 136, a pressurization spring 140, a dome-like metal plunger 144, and a drive spring 148.

The flexible needle cover 112 provides patient and device safety by protecting at least one needle 152 (described in greater detail below) and providing a sterile barrier. The needle cover 112, protects the needle 152 during device manufacture, protects the patient prior to use, and provides a sterility barrier at any point prior to removal. According to one embodiment, the needle cover 112 is attached via a press fit with a needle manifold in which the at least one needle 152 is disposed. Additionally, according to one embodiment, a needle opening 156 (described in greater detail below) of the safety mechanism 108 is shaped to closely correspond to a perimeter of the needle cover 112.

As shown, for example, in FIGS. 2, 3, 5, 6, 8, 10, and 12, the reservoir subassembly 120 includes a reservoir 160, a reservoir dome seal 164, a valve 168, at least one needle 152, and at least one channel 172 (see, for example, FIG. 8) disposed between the valve 168 and the needle 152 and creating a flow path therebetween. The reservoir 160 includes a dome 176. Additionally, the reservoir subassembly 120 includes the removable needle cover 112 to selectively cover the at least one needle 152. According to one embodiment, the reservoir subassembly 120 also includes a reservoir arm seal 180, covering the channel 172. Preferably, the needle 152 includes a needle manifold and a plurality of microneedles 152.

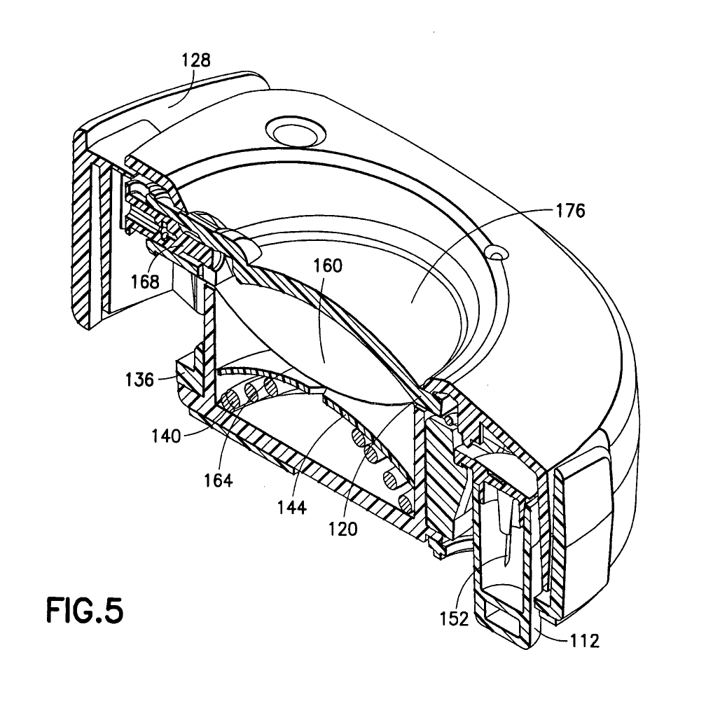

The reservoir dome seal (flexible film) 164 of the reservoir subassembly 120, as shown, for example, in FIG. 5, is disposed between the plunger 144 and the dome 176. Reservoir contents (for example, medicinal material) for the infusion device 100 are disposed in the space between the reservoir dome seal 164 and the dome 176. The combination of the reservoir dome seal 164, the dome 176, and the space therebetween defines a reservoir 160. The dome 176 is preferably transparent to permit viewing of the reservoir contents. The reservoir dome seal 164 can be made of non-distensible materials or laminates, such as metal-coated films or other similar substances. For example, one possible flexible laminate film that can be used in the reservoir dome seal 164 includes a first polyethylene layer, a second chemical layer as known to those skilled in the art to provide an attachment mechanism for a third metal layer which is chosen based upon barrier characteristics, and a fourth layer that includes polyester and/or nylon. By utilizing a metal-coated or metalized film in conjunction with a rigid portion (for example, dome 176), the barrier properties of the reservoir 160 are improved, thereby increasing or improving the shelf life of the contents contained within. For example, where a reservoir content includes insulin, the primary materials of contact in the reservoir 160 include linear, low-density polyethylene (LLDPE), low-density polyethylene (LDPE), cyclic olefin copolymer (COC) and Teflon. As described in greater detail below, the primary materials of contact in the remaining flow path of the reservoir contents may also include COC and LLDPE, as well as thermoplastic elastomer (TPE), medical grade acrylic, stainless steel, and a needle adhesive (e.g. a UV cured adhesive). Such materials that remain in extended contact with the contents of the reservoir 160 preferably pass ISO 10-993 and other applicable biocompatibility testing.

The reservoir subassembly 120 is further preferably able to be stored for the prescribed shelf life of the reservoir contents in applicable controlled environments without adverse effect to the contents, and is capable of applications in a variety of environmental conditions. Additionally, the barrier provided by the components of the reservoir subassembly 120 do not permit the transport of gas, liquid, and/or solid materials into or out of the contents at a rate greater than that allowable to meet the desired shelf life. In the embodiments shown above, the reservoir materials are capable of being stored and operated in a temperature range of approximately 34 to 120 degrees Fahrenheit and can have a shelf life of two or more years.

In addition to satisfying stability requirements, the reservoir subassembly 120 can further ensure operation by successfully passing any number of leak tests, such as holding a 30 psi sample for 20 minutes without leaking. Additional filling, storage and delivery benefits resulting from the configuration of the reservoir include minimized headspace and adaptability as described in greater detail below.

In one embodiment, the reservoir 160 is evacuated prior to filling. By evacuating the reservoir 160 prior to filling and having only a slight depression in the dome 176, headspace and excess waste within the reservoir 160 can be minimized. In addition, the shape of the reservoir can be configured to adapt to the type of energizing mechanism (for example, pressurization spring 140 and plunger 144) used. Additionally, using an evacuated flexible reservoir 160 during filling minimizes any air or bubbles within the filled reservoir 160. The use of a flexible reservoir 160 is also very beneficial when the infusion device 100 is subjected to external pressure or temperature variations, which can lead to increased internal reservoir pressures. In such case, the flexible reservoir 160 expands and contracts with the reservoir contents, thereby preventing possible leaks due to expansion and contraction forces.

Yet another feature of the reservoir 160 includes the ability to permit automated particulate inspection at the time of filling, or by a patient at the time of use. One or more reservoir barriers, such as the dome 176, can be molded of a transparent, clear plastic material, which allows inspection of the substance contained within the reservoir. The transparent, clear plastic material is preferably a cyclic olefin copolymer that is characterized by high transparency and clarity, low extractables, and biocompatibility with the substance contained in the reservoir 160. A suitable material is available from Zeon Chemicals, L.P., of Louisville, Ky. under the designation “BD CCP Resin,” and is listed by the U.S. Food and Drug Administration and DMF No. 16368. In such applications, the reservoir 160 includes minimal features that could possibly obstruct inspection (i.e. rotation during inspection is permitted).

Channel arm 172 is provided in the form of at least one flexible arcuate arm extending from the valve 168 to the needle manifold or microneedles 152. The arcuate arm has a groove 174 (see, for example, FIG. 2) formed therein. To provide a fluid path between valve 168 and the needle manifold or microneedles 152, the reservoir arm seal 180 covers the groove 174. The fluid path (disposed in channel arm 172—shown, for example, in FIG. 8) between the reservoir 160 and the microneedles 152 is constructed of materials similar or identical to those described above for the reservoir 160. For example, channel arm 172 may be constructed of the same material as the dome 160 and the reservoir arm seal 180 may constructed of the same material as the reservoir dome seal 164. According to one embodiment, both channel arms 172 are employed as fluid paths between the valve 168 and the needle manifold or microneedles 152. According to another embodiment, only one of the channel arms 172 is employed as a fluid path, and the remaining channel arm 172 provides structural support. In such an embodiment, the groove 174 extends fully from the valve 168 to the needle manifold or microneedles 152 only in the channel arm 174 that will be employed as the fluid path.

The channel arm 172 must be sufficiently flexible to withstand the force of activation. Contrasting the position of the channel arm 172 in FIGS. 2 and 8, the channel arm 172 (covered by reservoir arm seal 180 in FIG. 2, which is removed in FIG. 8 for clarity) elastically deforms when the microneedles 152 are driven into the patient's skin (described in greater detail below). During such deformation, the channel arm 172 must maintain the integrity of the fluid path between the valve 168 and the needle manifold or microneedles 152. Additionally, the materials for the channel arm 172 satisfy numerous biocompatibility and storage tests. For example, as shown in Table 1 below, where an infusion device content includes insulin, the primary materials of contact in the reservoir 160 include linear, low-density polyethylene, cyclic olefin copolymer, and Teflon, and can also include a transparent, clear plastic. The primary materials of contact in the remaining flow path (channel 62) between the reservoir 160 and the microneedles 152 of the needle manifold include COC and/or medical grade acrylic, LLDPE, TPE, and stainless steel, as well as the needle adhesive.

TABLE 1

| Path Component | Material |
|---|---|
| Reservoir | Polyethylene, cyclic olefin copolymer, |
| | and/or Teflon |
| Reservoir Dome Seal | Metal-coated film, such as |
| | polyethylene, aluminum, polyester, |
| | and/or nylon with a chemical tie layer |
| Valve | TPE |
| Needle Manifold | COC and/or medical grade acrylic |
| Needle adhesive | UV-cured adhesive |
| Microneedle | Stainless steel |

More specifically, the microneedles 152 can be constructed of stainless steel, and the needle manifold can be constructed of polyethylene and/or medical grade acrylic. Such materials, when in extended contact with the contents of the reservoir, preferably pass ISO 10-993 biocompatibility testing.

The valve 168, disposed between the reservoir 160 and the channel 172, selectively permits and restricts fluid flow between the reservoir 160 and the channel 172. The valve 168 moves between a pre-activated position (shown, for example, in FIGS. 2, 3, and 6) and an activated position (shown, for example, in FIGS. 8-10). When in the activated position, the valve permits fluid flow between the reservoir 160 and the channel 172, and therefore to the needle manifold and microneedles 152.

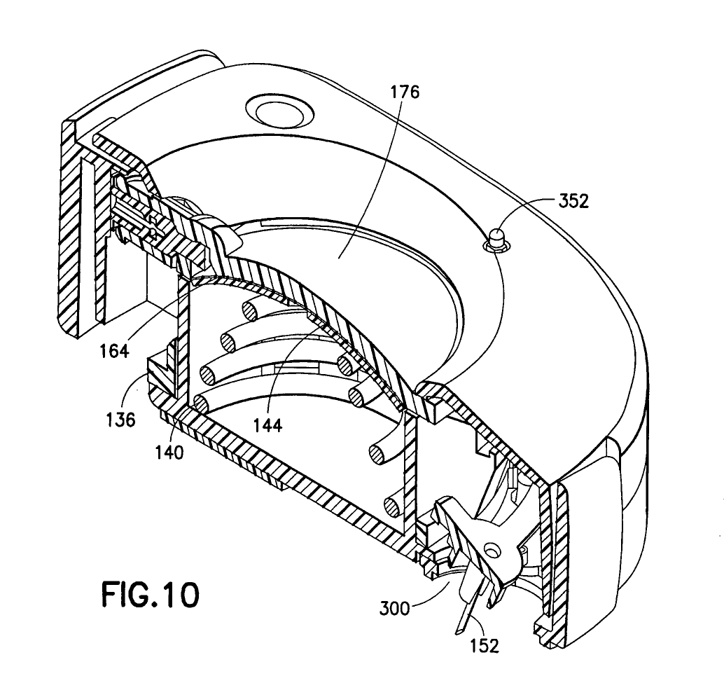

In use, the valve 168 will eventually be pushed into the activated position by the movement of the activator button 128, best illustrated by the movement of the valve 168 between FIGS. 5 and 10. As shown in FIG. 10, the movement of the valve 168 advances the enlarged distal end of the valve 168, thereby permitting the drug to flow from the reservoir 160 into the channel 172 and down the fluid path to the needle manifold.

The embodiment described above includes at least one needle 152, or microneedle 152, but may contain several, such as the two illustrated microneedles 152. Each microneedle 152 is preferably at least 31 gauge or smaller, such as 34 gauge, and is anchored within a patient needle manifold that can be placed in fluid communication with the reservoir 160. The microneedles 152, when more than one is included in the infusion device 100, can also be of differing lengths, or gauges, or a combination of both differing lengths and gauges, and can contain one or more ports along a body length, preferably located near the tip of the microneedle 152 or near the tip bevel if any of the microneedles 152 has one.

According to one embodiment, the gauge of the microneedles 152 governs the delivery rate of reservoir contents of the infusion device 100. The use of multiple 34 gauge microneedles 152 to deliver the reservoir contents is practical when the infusion occurs over a longer period than typically associated with an immediate syringe injection requiring a much larger cannula, or needle. In the disclosed embodiments, any microneedles 152 that target either an intradermal or subcutaneous space can be used, but the illustrated embodiments include intradermal microneedles 152 of between 1 and 7 mm in length (i.e., 4 mm). The arrangement of the microneedles 152 can be in a linear or nonlinear array, and can include any number of microneedles 152 as required by the specific application.

As noted above, the microneedles 152 are positioned in a needle manifold. In the needle manifold, at least one fluid communication path, or channel 172, is provided to each microneedle 152. The manifold may simply have a single path to one or more microneedles 152, or may provide multiple fluid paths or channels routing the reservoir contents to each microneedle 152 separately. These paths or channels may further comprise a tortuous path for the contents to travel, thereby affecting fluid pressures and rates of delivery, and acting as a flow restrictor. The channels or paths within the needle manifold can range in width, depth and configuration depending upon application, where channel widths are typically between about 0.015 and 0.04 inch, preferably 0.02 inch, and are constructed to minimize dead space within the manifold.

According to one embodiment, the reservoir subassembly 120 has a pair of holes 184 and 188 to aid registration of the reservoir subassembly 120 with respect to the bottom enclosure 104. First and second posts 192 and 196 (described in greater detail below) of the bottom enclosure 104 are inserted through the respective holes 184 and 188.

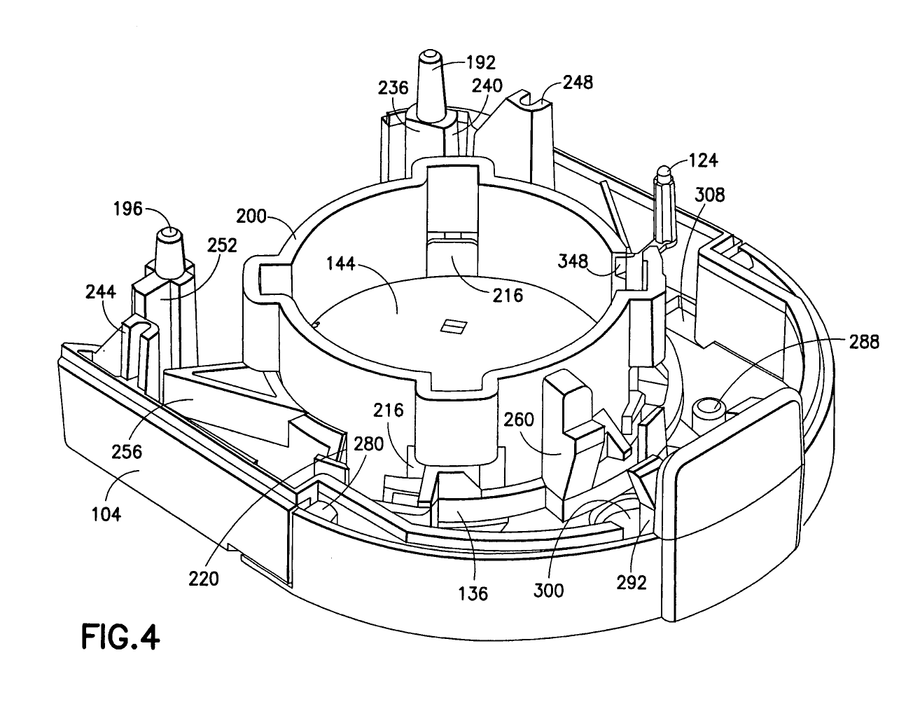

In exploded views with the reservoir subassembly 120 removed, FIGS. 4, 7, and 9 illustrate that bottom enclosure 104 includes a substantially cylindrical housing 200 in which pressurization spring 140 and plunger 144 are disposed. According to one embodiment, cylindrical housing 200 includes a plurality of recessed channels 204 to guide a respective plurality of legs 208 and feet 212 of the plunger 144 as the plunger translates within the housing 200. Collectively, a leg 208 and a foot 212 constitute a plunger tab 214. As shown in FIGS. 4, 7, and 9, for example, the recessed channels 204 extend only part of the way down the cylindrical housing 200 from a top thereof. Below the recessed channels 204, there are openings 216 through which the feet 212 of plunger 144 can extend outside of the cylindrical housing 200. The openings 216 are substantially L-shaped with horizontal portions at the base of the cylindrical housing 200, and a vertical portion substantially aligned with the recessed channels 204.

When the infusion device 100 is in the pre-activated state, the pressurization spring 140 is compressed by the plunger 144 (as shown, for example, in FIGS. 4-6), and the feet 212 of the plunger 144 are substantially disposed in the horizontal portions of the openings 216. The force of the pressurization spring 140 biases the feet 212 of the plunger 144 against a top of the horizontal portions of the openings 216 (i.e., a ledge of the cylindrical housing 200). Together, as described in greater detail below, the pressurization spring 140 and the plunger 144 form a pressurization system to pressurize the reservoir 160 when the infusion device 100 is activated.

As described in greater detail below, the rotor 136 rotates around the base of the cylindrical housing 200 between a pre-activated position (illustrated, for example, in FIGS. 2-4) and an activated position (illustrated, for example, in FIGS. 8-10). When the rotor 136 rotates from the pre-activated position to the activated position, at least one foot engaging surface 220 (shown, for example, in FIG. 4) of the rotor 136 engages at least one of the feet 212 of the plunger 144 and rotates the plunger 144 so that the feet 212 align with the vertical portions of the openings 216 and the recessed channels 204. At this point, the pressurization spring 140 moves the plunger 144 upward with the feet 212 being guided by the raised channels 204.

The pressurization spring 140 is included in the infusion device 100 to apply an essentially even force to the reservoir 160, to force the contents from the reservoir 160. The pressurization spring 140 is used to store energy that, when released, pressurizes the reservoir 160 at the time of use. The pressurization spring 140 is held in a compressed state by engagement between feet 212 of the plunger 144 and the cylindrical housing 200. This engagement prevents the pressurization spring 140 from putting stress on a film (to be described later) of the reservoir 160 or any remaining device components (other than the bottom enclosure 104 and the plunger 144) during storage. The plunger 144 is sufficiently rigid to resist spring tension and deformation, and should not fail under normal load.

As noted above, when the rotor 136 rotates from the pre-activated position to the activated position, the rotor 136 engages at least one of the feet 212 of the plunger 144 and rotates the plunger 144 to align the feet 212 with the vertical portions of the openings 216 and the recessed channels 204. The compressed pressurization spring 140, then moves the plunger 144 upward, and in doing so, exerts a force on the film of the reservoir 160. The pressurization spring 140 can be configured to preferably create a pressure within the reservoir 116 of from about 1 to 50 psi, and more preferably from about 2 to about 25 psi for intradermal delivery of the reservoir contents. For sub-cutaneous injection or infusion, a range of about 2 to 5 psi may be sufficient.

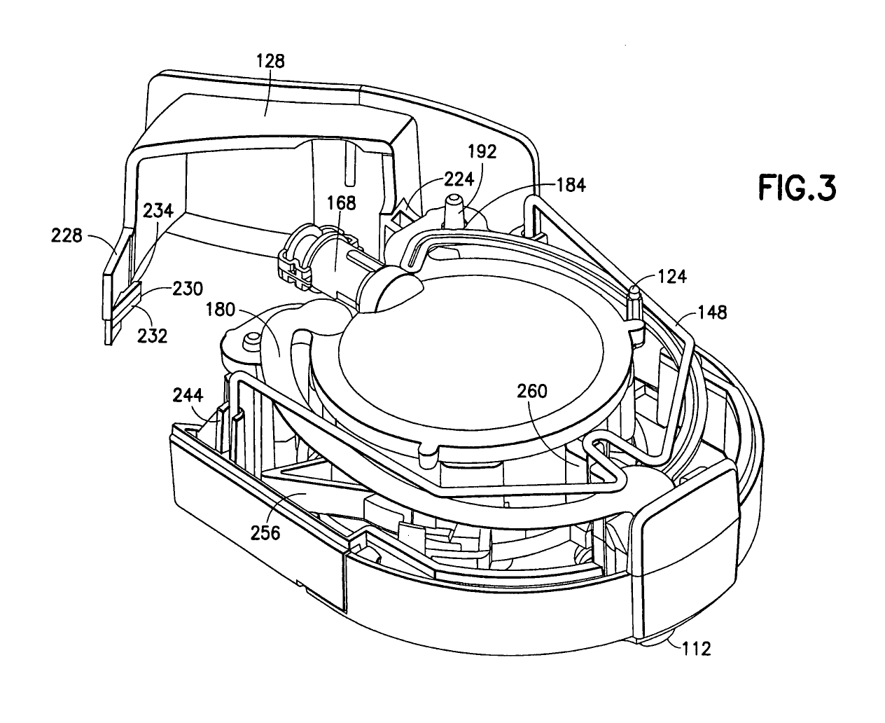

According to one embodiment, the activator button 128 includes the patient interface surface 132 that the patient presses to activate the infusion device 100. The activator button 128 also includes a hinge arm 224 and an activation arm 228 (both shown, for example, in FIG. 3). The hinge arm 224 of the activator button 128 includes a cylindrical portion with an opening. The activation arm 228 includes a tab 230 (see, for example, FIG. 3). According to one embodiment, the tab 230 includes a bearing surface 232 and a locking surface 234 disposed adjacent to the cantilevered end of the bearing surface 232. According to one embodiment, the tab 230 forms an acute angle with a main portion of the activation arm 228.

The first post 192, disposed on the bottom enclosure 104, extends upwardly therefrom. According to one embodiment (as shown, for example, in FIGS. 4 and 7), a base of the first post 192 includes a pair of flat sides 236 and a pair of rounded sides 240. Additionally, as shown, for example, in FIGS. 4 and 7, the second post 196 and first and second drive spring bases 244 and 248 extend upwardly from the bottom enclosure 104. As will be described in greater detail below, the first and second drive spring bases 244 and 248 anchor respective ends of drive spring 148. The first drive spring base 244 is disposed adjacent to the second post 196 with a space therebetween.

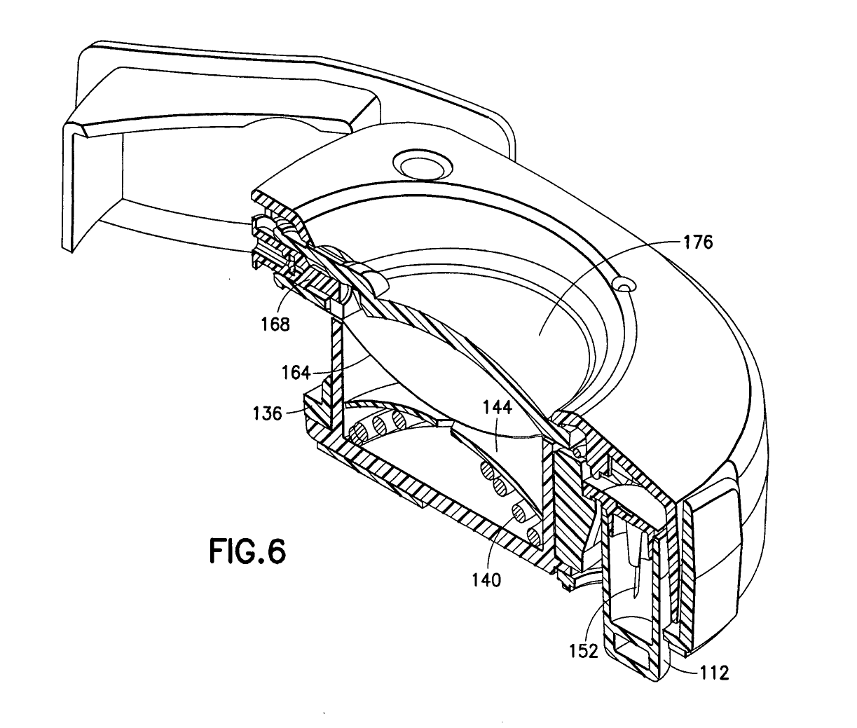

According to one embodiment, FIGS. 3 and 6 illustrate the positioning of the activator button 128 with respect to the bottom enclosure 104, for assembly of the activator button 128. In this position, the opening of the cylindrical portion of the hinge arm 224 allows the activator button 128 to slide horizontally (passing the flat sides 236) and engage the first post 192. The hinge arm 224 (and therefore the activator button 128) can then rotate about the first post 192. As the activation arm 228 passes into the space between the second post 196 and the first drive spring base 244, at least one of the tab 230 and the activation arm 228 elastically deforms until a cantilevered end of the bearing surface 232 of tab 230 passes a retaining face 252 of the second post 196. The passage of the cantilevered end of the bearing surface 232 of tab 230 past the retaining face 252 (see, for example, FIG. 4) of the second post 196 and the engagement of the locking surface 234 of tab 230 with the retaining face 252 provides an audible click and tactile feedback conveying that the activator button 128 is in the pre-activated position.

Referring back to FIGS. 2-4, and 7-9, rotor 136 additionally includes an activation projection 256 and a drive spring holder 260. The activation arm 228 of the activator button 128 engages the activation projection 256 when a patient depresses the activator button 128, thereby rotating the rotor 136 from the pre-activated position to the activated position.

The drive spring holder 260 maintains the drive spring 148 in a pre-activated position when the rotor 136 is in the pre-activated position. As noted previously, the first and second drive spring bases 244 and 248 anchor opposing ends of the drive spring 148. At approximately a midpoint of the drive spring 148, there is a substantially U-shaped projection as shown, for example, in FIGS. 2 and 3, for engagement with the drive spring holder 260 of the rotor 136. Accordingly, when the rotor 136 is in the pre-activated position and the drive spring 148 engages the drive spring holder 260, the drive spring 148 is maintained in a tensile state. And when the drive spring holder 260 releases the drive spring 148 (i.e., when the rotor rotates from the pre-activated position to the activated position as illustrated, for example, in FIGS. 8-10), the drive spring 148 drives the microneedles 152 to extend outside of the infusion device 100 through an opening 300 in the bottom enclosure 104 (and through an opening in the safety mechanism 108 described in greater detail below).

Thus, as will be described in greater detail below, the activation and energizing of the infusion device 100 that is accomplished in a single multi-function/step process includes depression of the activator button 128 by a patient, and rotation of the rotor 136 due to engagement between the activation arm 228 of the activator button 128 and the activation projection 256 of the rotor 136. As described above, the rotation of the rotor 136 rotates and releases the plunger 144 to pressurize the fluid within the reservoir 160. Additionally, the rotation of the rotor 136 releases the drive spring 148 from the drive spring holder 260, thereby driving the microneedles 152 to extend outside of the infusion device 100. The single multi-function/step process also includes movement of the valve 168 from the pre-activated position to the activated position due to the activator button 128 engaging and moving the valve 168 when the activator button 128 is depressed, thereby commencing fluid flow between the reservoir and the microneedles 152 via the channel 172.

As noted above, the patch-like infusion device 100 also includes a safety mechanism 108. To prevent inadvertent or accidental needle stick injuries, prevent intentional re-use of the device, and to shield exposed needles, the locking needle safety mechanism 108 is provided. The safety mechanism 108 automatically activates immediately upon removal of the infusion device 100 from the skin surface of the patient. According to one embodiment described in greater detail below, a flexible adhesive pad 264 adheres to a bottom portion of the bottom enclosure 104 and a bottom portion of the safety mechanism 108. The adhesive pad 264 contacts with the patient's skin and holds the infusion device 100 in position on the skin surface during use. As shown, for example, in FIGS. 11 and 12, upon removal of the infusion device 100 from the skin surface, the safety mechanism 108 extends to a position shielding the microneedles 152. When fully extended, safety mechanism 108 locks into place and prevents accidental injury or exposure to the patient needles.

In general, a passive safety system is most desirable. This allows the device to be self-protecting in case of accidental removal or if the patient forgets that there is a safety step. Because one typical use for this infusion device 100 is to provide human growth hormone, which is usually given in the evening, it can be expected that patients that wear the device (such as children) may actually wear them overnight, even though the delivery may be expected to take less than 10 minutes. Without a passive system, if the infusion device 100 falls off, the microneedles 152 could re-stick the patient or a caregiver. The solution is to either limit the activities during use, or include a passive safety system.

With respect to safety systems, there are typically three options. A first option is to retract the needles 152 into the device. A second option is to shield the needles 152 to remove access, and a third option is to destroy the needles 152 in a way that prevents needle stick injuries. Other systems, such as active systems, utilize manual shielding and/or destruction, or manual release of safety features with an additional button push or similar action. A detailed description of passive safety embodiments of the present invention is provided below.

One safety embodiment of the present invention is a passive, fully enclosed pull-out design embodiment, such as safety mechanism 108. FIGS. 5, 10, and 12 are perspective cutaway views of the infusion device 100 that illustrate the safety mechanism 108 prior to activation, subsequent to activation, and subsequent to deployment of the safety mechanism 108, respectively.

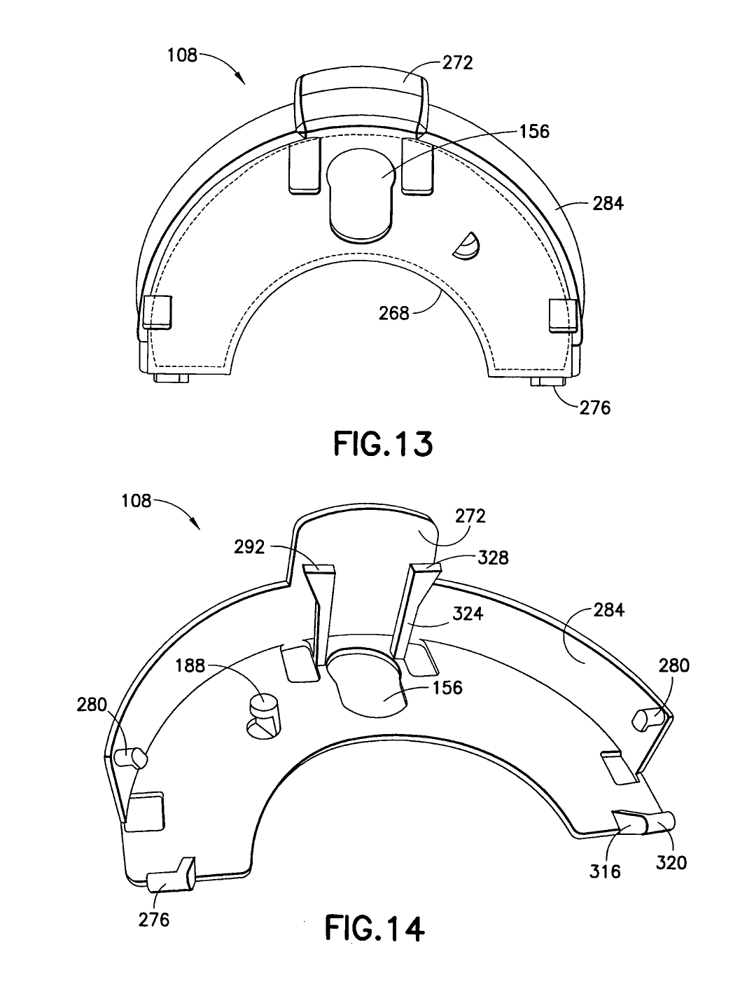

When the infusion device 100 is removed from the skin, the flexible adhesive pad 264 (attached to both the bottom surface of the bottom enclosure 104 and the bottom surface of the safety mechanism 108) will pull the safety mechanism 108 out and lock it into place before the adhesive pad 264 releases the skin surface. In other words, the force required to remove the adhesive pad from the skin surface is greater than that required to deploy the safety mechanism 108. According to one embodiment, the safety mechanism 108, as shown, for example, in FIG. 13, includes a flat surface portion 268 that is in contact with the patient's skin. The flat surface 268 is where a portion of adhesive pad 264 (shown as a dotted line in FIG. 13) is affixed to safety mechanism 108 such that when the infusion device 100 is removed by the patient from the skin, the adhesive pad 264 will act to deploy the safety mechanism 108 from the infusion device 100, thereby shielding the microneedles 152, which otherwise would be exposed upon removal of the infusion device 100 from the patient. When the safety mechanism 108 is fully extended, the safety mechanism 108 locks into place and prevents accidental injury or exposure to the microneedles 152.

According to one embodiment, the adhesive pad 264 is provided in substantially two parts, one on the bulk of the bottom surface of the bottom enclosure 104, and one on the bottom surface of the safety mechanism 108. When the infusion device 100 is removed, the two patches move independently and the safety mechanism 108 is rotatable with respect to the bottom enclosure 104. According to another embodiment, the two parts are formed as a unitary, flexible adhesive pad 264 with one part being disposed on the on the bulk of the bottom surface of the bottom enclosure 104, and one part disposed on the bottom surface of the safety mechanism 108.

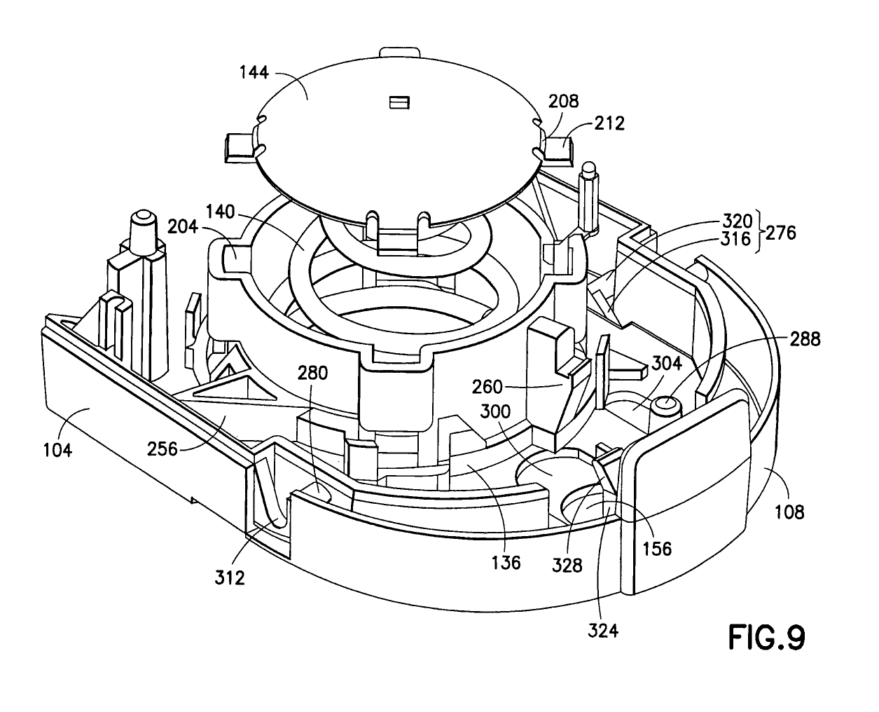

According to one embodiment, the safety mechanism 108 is a stamped metal part. According to another embodiment, the safety mechanism 108 is made of substantially the same material as the bottom enclosure 104. As shown in FIG. 14, the safety mechanism 108 includes a front shield 272, a pair of insertion tabs 276 disposed at a rear portion of the safety mechanism 108, a pair of pivot tabs 280 disposed, respectively, at upper rear ends of a rim portion 284 of the safety mechanism 108, a guide post 288 extending upwardly from a substantially flat bottom inner surface of the safety mechanism 108, and locking posts 292 also extending upwardly from the bottom inner surface of the safety mechanism 108. Front shield 272 extends above the rim portion 284 to shield the patient from the microneedles 152 when the safety mechanism 108 is deployed. The guide post 288 includes a cutout therein to engage a safety retaining projection 296 of the rotor 136 (shown, for example, in FIGS. 7 and 9) when the rotor 136 is in the pre-activated position, to prevent the safety mechanism 108 from deploying prior to activation of the infusion device 100.

Additionally, as noted above, the safety mechanism 108 includes the needle opening 156. Prior to deployment of the safety mechanism 108, the needle opening 156 at least partially overlaps the opening 300 in bottom enclosure 104 to provide space for movement of the microneedles 152. The locking posts 292 are respectively disposed adjacent to front side edges of the needle opening 156. The bottom enclosure 104 includes a guidepost opening 304 (shown, for example, in FIGS. 7 and 9), a pair of insertion tab openings 308 (one of which is shown, for example, in FIG. 4) disposed adjacent to opposing side edges of the bottom enclosure 104, and a pair of pivot rests 312 disposed on opposing sides of the bottom enclosure 104 (shown, for example, in FIGS. 7 and 9).

Referring again to FIG. 14, insertion tabs 276 each include a connecting portion 316 and an extending portion 320. According to one embodiment, the connecting portions 316 extend from the bottom inner surface of the safety mechanism 108 toward a rear of the infusion device 100 at a non-perpendicular angle with respect to the bottom inner surface of the safety mechanism 108. Extending portions 320 each extend substantially perpendicularly from the extending portions 320 toward respective outer sides of the safety mechanism 108. To assemble the safety mechanism 108 to the bottom enclosure 104, safety mechanism 108 is held at an approximately 45° angle with respect to the bottom enclosure 104 and the insertion tabs 276 are inserted through the insertion tab openings 308. The safety mechanism 108 is then rotated to a position such that the guidepost 288 is inserted through the guidepost opening 304 and the bottom inner surface of the safety mechanism 108 is substantially parallel and in contact with the bottom surface of the bottom enclosure 104.

Referring again to FIGS. 7 and 9, although these views illustrate the rotor 136 in the activated position, the exploded nature of FIGS. 7 and 9 is convenient to illustrate this stage of the assembly of the safety mechanism 108 to the bottom enclosure 104. It will be understood, however, that the safety mechanism 108 should be assembled to the bottom enclosure prior to activation. Subsequent to the upward rotation of the safety mechanism 108, as shown in FIG. 4, safety mechanism 108 translates rearward with respect to the bottom enclosure 104 such that pivot tabs 280 clear respective front edges of the pivot rests 312 and are disposed above the pivot rests 312, the locking posts 292 are disposed adjacent to side edges of the opening 300 of the bottom enclosure 104, and the safety retaining projection 296 of the rotor 136 engages the guide post 288.

Returning to FIG. 14, each of the locking posts 292 includes a post extending portion 324 extending substantially perpendicular from the flat bottom inner surface of the safety mechanism 108, and a wedge portion 328 disposed at an end of the post extending portion 324. As a height of the wedge portion 328 increases with respect to the bottom inner surface of the safety mechanism 108, a width of the wedge portion 328 increases.

As the safety mechanism 108 deploys and rotates downward with respect to the bottom enclosure 104, the wedge portions 328 act against respective side edges of the openings 180 of the bottom enclosure 104, causing the locking posts 192 to deform elastically toward one another. As the safety mechanism 108 is fully deployed, the tabs 280 become seated in pivot rests 312. Additionally, top edges of the wedge portions 328 pass bottom edges of the opening 300 and the locking posts 292 snap back to their substantially un-deformed states, providing an audible click and tactile feedback communicating that the safety mechanism 108 is fully deployed, and therefore, that the microneedles 152 are covered. Returning to FIGS. 11 and 12, once the safety mechanism 108 is fully deployed and the locking posts 292 have snapped back to their substantially un-deformed states, the top edges of the wedge portions 328 engage the bottom surface of the bottom enclosure 104 adjacent to the opening 300, thereby preventing the safety mechanism 108 from rotating upward with respect to the bottom enclosure 104 and exposing the microneedles 152. Additionally, as noted above, front shield 272 shields the patient from the microneedles 152.

Accordingly, the safety mechanism 108 is a passive safety embodiment provided as a single part and provides a good lock that will not crush under human loads. With this passive safety mechanism, no additional forces are applied to the skin during injection, and the microneedles 152 are safely held within the infusion device 100 after use.

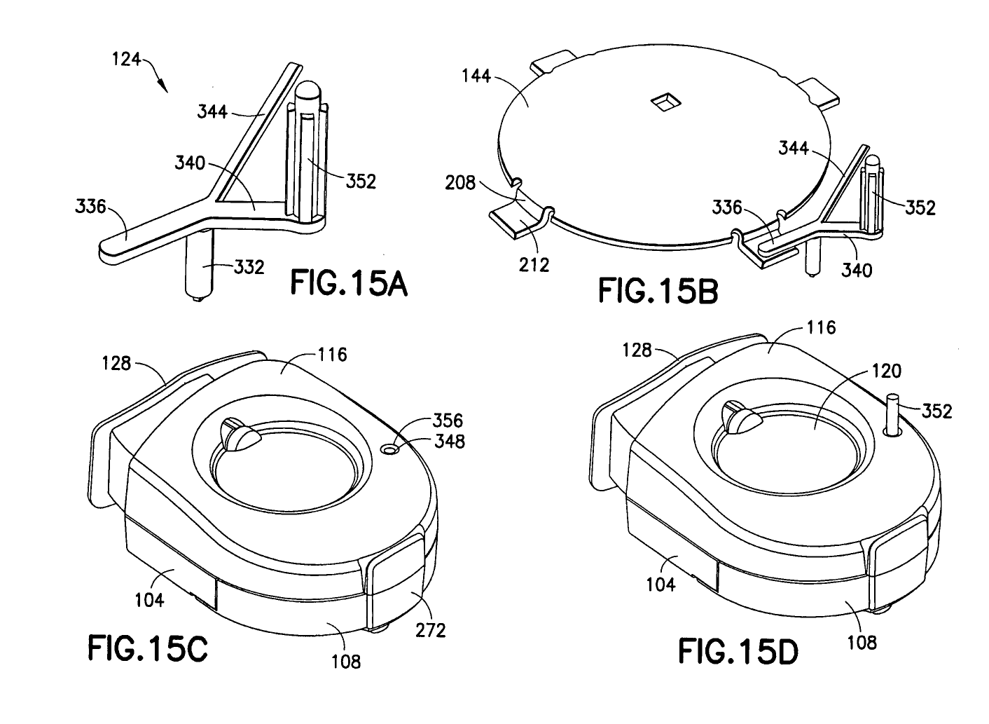

After use of the infusion device 100, the patient can once again inspect the device to ensure the entire dose was delivered. In this regard, as shown in FIGS. 15A-D, the infusion device 100 includes the end-of-dose indicator (EDI) 124. The EDI 124 includes a main body 332 and first and second arms 336 and 340 extending substantially horizontally with respect to a top of the main body 332.

The EDI 124 also includes a spring arm 344 that curves upwardly from the top of the main body 332. According to one embodiment, the spring arm 344 pushes against a bottom side of the reservoir subassembly 120, elastically biasing the EDI 124 toward the bottom enclosure 104, to ensure that the EDI 124 does not move freely out of the infusion device 100, for example, during shipping and handling of the infusion device 100.

Returning to FIG. 4, the main body 332 is disposed in an EDI channel 348 and translates substantially vertically therein. The EDI channel adjacent to one of the recessed channels 204 that guides legs 208 and feet 212 of plunger 144. The first arm 336 extends across a top of this recessed channel 204.

Returning to FIG. 15A, a vertical extrusion 352 extends upwardly from an end of the second arm 340. When the reservoir contents have been delivered, the vertical extrusion extends through an EDI opening 356 (see, for example, FIG. 15C) in the top enclosure 116 to communicate that the end of the dose has been reached. According to one embodiment, the EDI 124 is formed as a one-piece construction.

As shown in FIG. 15B, as the plunger 144 travels upwardly in the cylindrical housing 200 due to the pressurization spring 140 subsequent to activation, one of the feet 212 of the plunger 144 contacts the first arm of the EDI 124. The foot 212 lifts the EDI 124 upward, overcoming the bias of the spring arm 344, and causing the vertical extrusion 352 to increasingly extend through the EDI opening 356 during delivery of the reservoir contents. Referring back to FIG. 10, vertical extrusion 352 partially extends from the infusion device 100. Once the delivery of the reservoir contents is complete and the plunger has achieved its full stroke, the vertical extrusion 352 is fully extended, as shown in FIG. 15D. Thus, the EDI 124 employs the linear movement of the plunger 144 to generate linear movement of the EDI 124 that is visible outside of the infusion device 100 thereby communicating the delivery of the reservoir contents.

FIG. 16 illustrates an embodiment of an infusion device 400 with an injection port 404. The injection port provides access to an evacuated or partially-filled reservoir 408, so that the patient can inject a substance or combination of substances into the reservoir prior to activation. Alternatively, a pharmaceutical manufacturer or pharmacist could employ the injection port 404 to fill the infusion device 400 with a substance or combination of substances prior to sale. In substantially all other respects, the infusion device 400 is similar to the previously-described infusion device 100.

Operation of the infusion device 100 will now be described. The embodiments of the present invention described above preferably include a push-button (activator button 128) design wherein the infusion device 100 can be positioned and affixed to a skin surface, and energized and/or activated by pressing the activator button 128. More specifically, in a first step, the patient removes the device from a sterile packaging (not shown), removes a cover (not shown) of the adhesive pad 264. The patient also removes the needle cover 112. Upon removal of the infusion device 100 from the package and prior to use (see, for example, FIGS. 1, 2, 4, and 5), the infusion device 100 in the pre-activated state allows the patient to inspect both the device and the contents therein, including inspection for missing or damaged components, expiration dates(s), hazy or color-shifted drugs, and so forth.

The next step is the positioning and application of the infusion device 100 to the patient's skin surface. Like a medicinal patch, the patient firmly presses the infusion device 100 onto the skin. One side of the adhesive pad 264 adheres to a bottom surface of the bottom enclosure 104 and a bottom surface of the safety mechanism 108, and the opposing side of the adhesive pad 264 secures the infusion device 100 to the skin of the patient. These bottom surfaces (of the bottom enclosure 104 and the safety mechanism 108) can be flat, contoured, or shaped in any suitable fashion and the adhesive pad 264 is secured thereon. According to one embodiment, prior to shipping, the cover of the adhesive pad 264, such as a film, is applied to the patient-side of the adhesive pad 264 to preserve the adhesive during shipping. As noted above, prior to use, the patient peels back the adhesive cover, thereby exposing the adhesive pad 264 for placement against the skin.

After removing the adhesive cover, the patient is able to place the infusion device 100 against the skin and press to ensure proper adhesion. As noted above, once properly positioned, the device is activated by depressing the activator button 128. This activation step releases plunger 144 and the pressurization spring 140, allowing a plunger 144 to press against the flexible film (reservoir dome seal 164) of the reservoir 160, thereby pressurizing the reservoir. This activation step also serves to release the drive spring 148 from the drive spring holder 260 of the rotor 136, thereby driving the microneedles 152 to extend outside the infusion device 100 (through the opening 300 in the bottom enclosure 104 and the needle opening 156 of the safety mechanism 108) and seat the microneedles 152 within the patient. Further, the activation step opens the valve 168, establishing a fluid communication path between the reservoir 160 and the microneedles 152, via the channel 172 (see, for example, FIGS. 8-10). A significant benefit derives from the ability to achieve each of these actions in a single push-button operation. Additionally, another significant benefit includes the use of a continuous fluid communication path comprised entirely within the reservoir subassembly 120.

Once activated, the patient typically leaves the infusion device 100 in position, or wears the device, for some period of time (such as ten minutes to seventy-two hours) for complete delivery of the reservoir contents. The patient then removes and discards the device with no damage to the underlying skin or tissue. Upon intentional or accidental removal, one or more safety features deploy to shield the exposed microneedles 152. More specifically, when the infusion device 100 is removed by the patient from the skin, the adhesive pad 264 acts to deploy the safety mechanism 108 from the infusion device 100, thereby shielding the microneedles 152, which otherwise would be exposed upon removal of the infusion device 100 from the patient. When the safety mechanism 108 is fully extended, the safety mechanism 108 locks into place and prevents accidental injury or exposure to the microneedles 152. The safety features, however, can be configured to not deploy if the activator button 128 has not been depressed and the microneedles 152 have not been extended, thereby preventing pre-use safety mechanism deployment. After use, the patient can once again inspect the device to ensure the entire dose was delivered. For example, the patient can view the reservoir interior through the transparent dome 176 and/or inspect the EDI 124.

The described embodiments are suitable for use in administering various substances, including medications and pharmaceutical agents, to a patient, and particularly to a human patient. As used herein, a pharmaceutical agent includes a substance having biological activity that can be delivered through the body membranes and surfaces, and particularly the skin. Examples, listed in greater detail below, include antibiotics, antiviral agents, analgesics, anesthetics, anorexics, antiarthritics, antidepressants, antihistamines, anti-inflammatory agents, antineoplastic agents, vaccines, including DNA vaccines, and the like. Other substances that can be delivered intradermally or subcutaneously to a patient include human growth hormone, insulin, proteins, peptides and fragments thereof. The proteins and peptides can be naturally occurring, synthesized or recombinantly produced. Additionally, the device can be used in cell therapy, as during intradermal infusion of dendritic cells. Still other substances which can be delivered in accordance with the method of the present invention can be selected from the group consisting of drugs, vaccines and the like used in the prevention, diagnosis, alleviation, treatment, or cure of disease, with the drugs including Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as hirulog, IgE suppressors, Insulin, insulinotropin and analogs, Insulin-like growth factors, Interferons, Interleukins, Leutenizing hormone, Leutenizing hormone releasing hormone and analogs, Low molecular weight heparin, M-CSF, metoclopramide, Midazolam, Monoclonal antibodies, Narcotic analgesics, nicotine, Non-steroid anti-inflammatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF-, and TNF-antagonist, the vaccines, with or without carriers/adjuvants, including prophylactics and therapeutic antigens (including but not limited to subunit protein, peptide and polysaccharide, polysaccharide conjugates, toxoids, genetic based vaccines, live attenuated, reassortant, inactivated, whole cells, viral and bacterial vectors) in connection with, addiction, arthritis, cholera, cocaine addiction, diphtheria, tetanus, HIB, Lyme disease, meningococcus, measles, mumps, rubella, varicella, yellow fever, Respiratory syncytial virus, tick borne japanese encephalitis, pneumococcus, streptococcus, typhoid, influenza, hepatitis, including hepatitis A, B, C and E, otitis media, rabies, polio, HIV, parainfluenza, rotavirus, Epstein Barr Virus, CMV, chlamydia, non-typeable haemophilus, moraxella catarrhalis, human papilloma virus, tuberculosis including BCG gonorrhoea, asthma, atheroschlerosis malaria, E-coli, Alzheimers, H. Pylori, salmonella, diabetes, cancer, herpes simplex, human papilloma and the like other substances including all of the major therapeutics such as agents for the common cold, Anti-addiction, anti-allergy, anti-emetics, anti-obesity, antiosteoporeteic, anti-infectives, analgesics, anesthetics, anorexics, antiarthritics, antiasthmatic agents, anticonvulsants, anti-depressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, sexual hypofunction and tranquilizers and major diagnostics such as tuberculin and other hypersensitivity agents as described in U.S. Pat. No. 6,569,143, entitled “Method of Intradermally Injecting Substances”, the entire content of which is expressly incorporated herein by reference.

Vaccine formulations which can be delivered in accordance with the system and method of the present invention can be selected from the group consisting of an antigen or antigenic composition capable of eliciting an immune response against a human pathogen, which antigen or antigenic composition is derived from HIV-1, (such as tat, nef, gp120 or gp160), human herpes viruses (HSV), such as gD or derivatives thereof or Immediate Early protein such as ICP27 from HSVI or HSV2, cytomegalovirus (CMV (esp Human) (such as gB or derivatives thereof), Rotavirus (including live-attenuated viruses), Epstein Barr virus (such as gp350 or derivatives thereof), Varicella Zoster Virus (VZV, such as gpl, II and IE63) or from a hepatitis virus such as hepatitis B virus (for example Hepatitis B Surface antigen or a derivative thereof), hepatitis A virus (HAV), hepatitis C virus and hepatitis E virus, or from other viral pathogens, such as paramyxoviruses: Respiratory Syncytial virus (RSV, such as F and G proteins or derivatives thereof), parainfluenza virus, measles virus, mumps virus, human papilloma viruses (HPV for example HPV6, 11, 16, 18), flaviviruses (e.g. Yellow Fever Virus, Dengue Virus, Tick-borne encephalitis virus, Japanese Encephalitis Virus) or Influenza virus (whole live or inactivated virus, split influenza virus, grown in eggs or MDCK cells, or whole flu virosomes or purified or recombinant proteins thereof, such as HA, NP, NA, or M proteins, or combinations thereof), or derived from bacterial pathogens such as Neisseria spp, including N. gonorrhea and N. meningitidis (for example capsular polysaccharides and conjugates thereof, transferrin-binding proteins, lactoferrin binding proteins, PilC, adhesins); S. pyogenes (for example M proteins or fragments thereof, C5A protease, lipoteichoic acids), S. agalactiae, S. mutans; H. ducreyi; Moraxella spp, including M. catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C), M. bovis, M. leprae, M. avium, M. paratuberculosis M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. Epidermidis; Enterococcus spp., including E. faecalis, E. faecium; Clostridium spp., including C. tetani (for example tetanus toxin and derivative thereof), C. botulinum (for example Botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. Burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. Hermsii; Ehrlichia spp., including E. equi and the agent of the Human Granulocytic Ehrlichiosis; Rickettsia spp, including R. rickettsii; Chlamydia spp., including C. Trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. Falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. Carinii; Trichomonas spp., including T. vaginalis; Schisostoma spp., including S. mansoni, or derived from yeast such as Candida spp., including C. albicans; Cryptococcus spp., including C. neoformans, as described in PCT Patent Publication No. WO 02/083214, entitled “Vaccine Delivery System”, the entire content of which is expressly incorporated herein by reference.

These also include other preferred specific antigens for M. tuberculosis, for example Tb Ra12, Tb H9, Tb Ra35, Tb38-1, Erd 14, DPV, MTI, MSL, mTTC2 and hTCC1. Proteins for M. tuberculosis also include fusion proteins and variants thereof where at least two, preferably three polypeptides of M. tuberculosis are fused into a larger protein. Preferred fusions include Ra12-TbH9-Ra35, Erd14-DPV-MTI, DPV-MTI-MSL, Erd14-DPV-MTI-MSL-mTCC2, Erd14-DPV-MTI-MSL, DPV-MTI- MSL-mTCC2, TbH9-DPV-MTI. Most preferred antigens for Chlamydia include for example the High Molecular Weight Protein (HWMP), ORF3, and putative membrane proteins (Pmps). Preferred bacterial vaccines comprise antigens derived from Streptococcus spp, including S. pneumoniae (for example capsular polysaccharides and conjugates thereof, PsaA, PspA, streptolysin, choline-binding proteins) and the protein antigen Pneumolysin (Biochem Biophys Acta, 1989,67,1007; Rubins et al., Microbial Pathogenesis, 25, 337-342), and mutant detoxified derivatives thereof. Other preferred bacterial vaccines comprise antigens derived from Haemophilus spp., including H. influenzae type B (“Hib”, for example PRP and conjugates thereof), non typeable H. influenzae, for example OMP26, high molecular weight adhesins, P5, P6, protein D and lipoprotein D, and fimbrin and fimbrin derived peptides or multiple copy variants or fusion proteins thereof. Derivatives of Hepatitis B Surface antigen are well known in the art and include, inter alia, PreS1, PreS2 S antigens. In one preferred aspect the vaccine formulation of the invention comprises the HIV-1 antigen, gp120, especially when expressed in CHO cells. In a further embodiment, the vaccine formulation comprises gD2t as hereinabove defined.

In addition to the delivery of substances listed above, the infusion device 100 can also be used for withdrawing a substance from a patient, or monitoring a level of a substance in the patient. Examples of substances that can be monitored or withdrawn include blood, interstitial fluid or plasma. The withdrawn substances can then be analyzed for analytes, glucose, drugs, and the like.

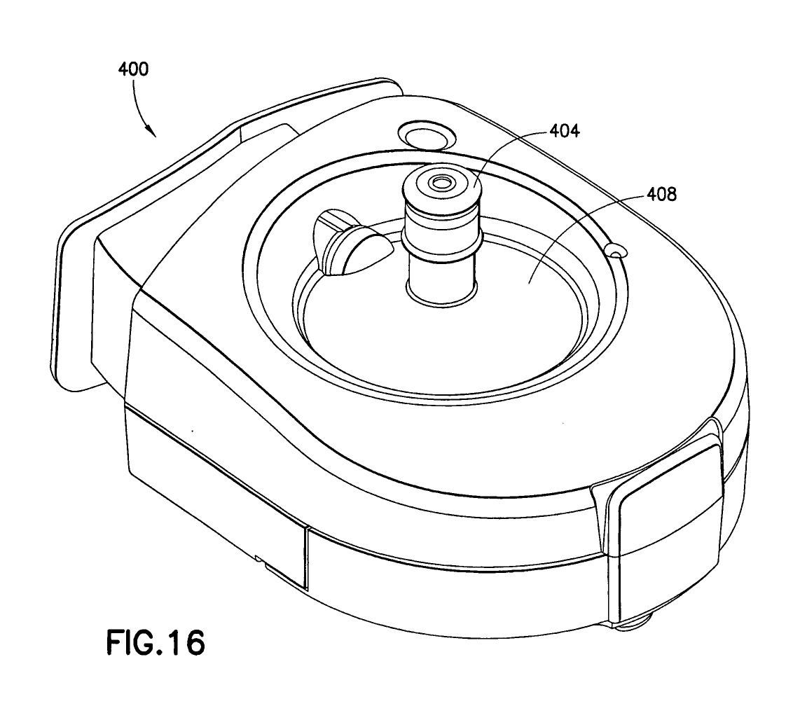

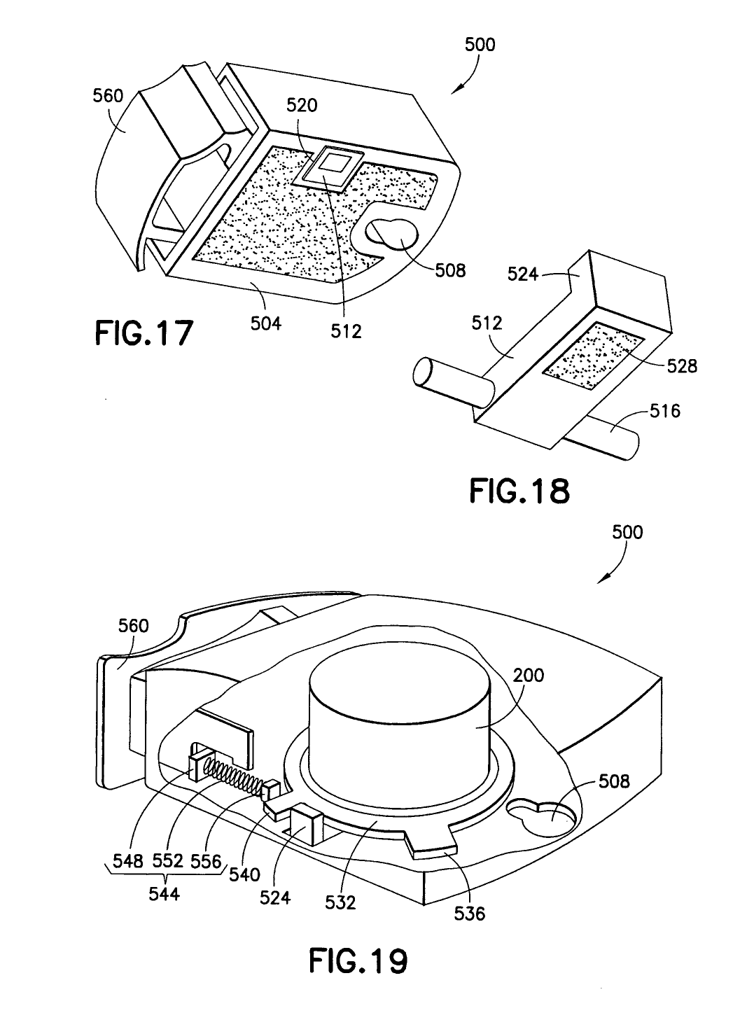

FIG. 17 illustrates an embodiment of an infusion device 500 with another embodiment of a safety mechanism. As shown in FIG. 17, the infusion device 500 includes a body 504, and a bottom of the body 504 includes a needle opening 508. According to one embodiment, the needle opening 508 has a shape corresponding to the needle cover 112. The body 504 also includes a blocker 512 that is movably connected to the body 504. More specifically, according to one embodiment, the blocker 512 is rotatably connected to the body 504.

As shown in FIG. 18, the blocker 512 includes a pair of blocker arms 516 extending therefrom. To assemble the blocker 512 to the body 504, blocker 512 inserted into a blocker opening 520 of the blocker body 504, such that the blocker arms 516 are snapped into fittings molded in the body 504 to rotatably connect the blocker 512 to the body 504. According to one embodiment, the blocker 512 is inserted into the blocker opening 520 from inside the body 504. According to another embodiment, the blocker 512 is inserted into the blocker opening 520 from outside the body 504.

The blocker 512 also includes a blocker post 524 extending from an end thereof and a blocker adhesive 528 disposed on an external surface (i.e., a patient surface) of the blocker 512. According to one embodiment, the blocker adhesive 528 is an adhesive pad. The blocker opening 520 is longitudinally sized to permit the blocker 512 to rotate away from the body 504 such that the blocker post 524 does not protrude into the body 504 beyond an internal bottom surface of the body 504. In other words, the longitudinal size of the blocker opening 520 must be at least slightly bigger than a longitudinal size of the blocker 512 so that the blocker post 524 may pass through the blocker opening 520.

As shown in FIG. 19, the infusion device 500 also includes a ring-like safety member or covering ring 532, which is rotatably disposed about the cylindrical housing 200 in which the plunger 144 travels. The safety member 532 includes a covering tab 536 extending therefrom for selectively covering the needle opening 508 and the blocking tab 540 extending therefrom for selectively bearing against the blocker 512. The safety member 532 is movable within the body 504 from a pre-deployed position (shown, e.g., in FIG. 19) to a covered position, in which the covering tab 536 covers the needle opening 508. The infusion device 500 additionally includes a biasing mechanism 544.

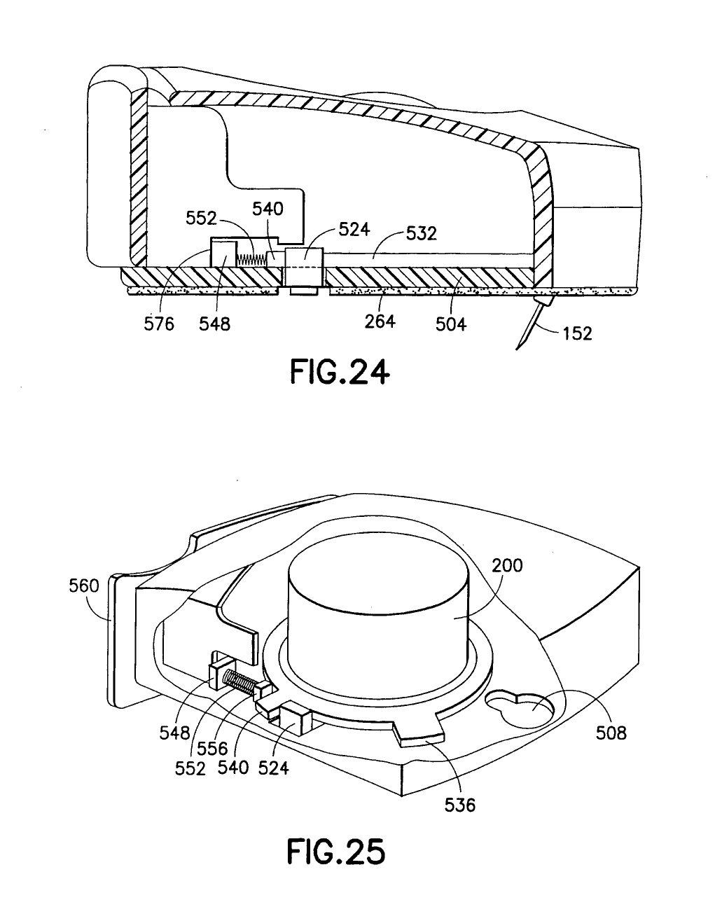

According to one embodiment, the biasing mechanism 544 includes a biasing member 548 and a biasing spring 552 connected thereto. As shown, for example, in FIGS. 21-23, according to one embodiment, the biasing spring 552 bears directly against the blocking tab 540. Additionally, as illustrated, for example, in FIGS. 19 and 24, according to another embodiment, the biasing mechanism 544 also includes a safety-bearing member 556 connected to the biasing spring 532 and disposed between the biasing spring 532 and the blocking tab 540. Further, the biasing spring 532 may be, for example, a coil spring or a leaf spring.

In one embodiment, the body 504 has a track disposed on an internal bottom surface thereof and the biasing member 548 has at least one foot that engages the track, such that the biasing member 548 is constrained vertically and moves smoothly along the track when a biasing bearing surface 576 of an activator button 560 (described in greater detail below) presses against the biasing member 548. In another embodiment, both the biasing member 548 and the safety bearing member 556 have at least one foot that engages the track, such that that the biasing member 548 and the safety bearing member 556 are constrained vertically and move smoothly along the track when the biasing bearing surface 576 of the activator button 560 presses against the biasing member 548. The movement of the safety bearing member 556 is induced by the compression of the biasing spring 552 when the biasing bearing surface 576 presses against the biasing member 548.

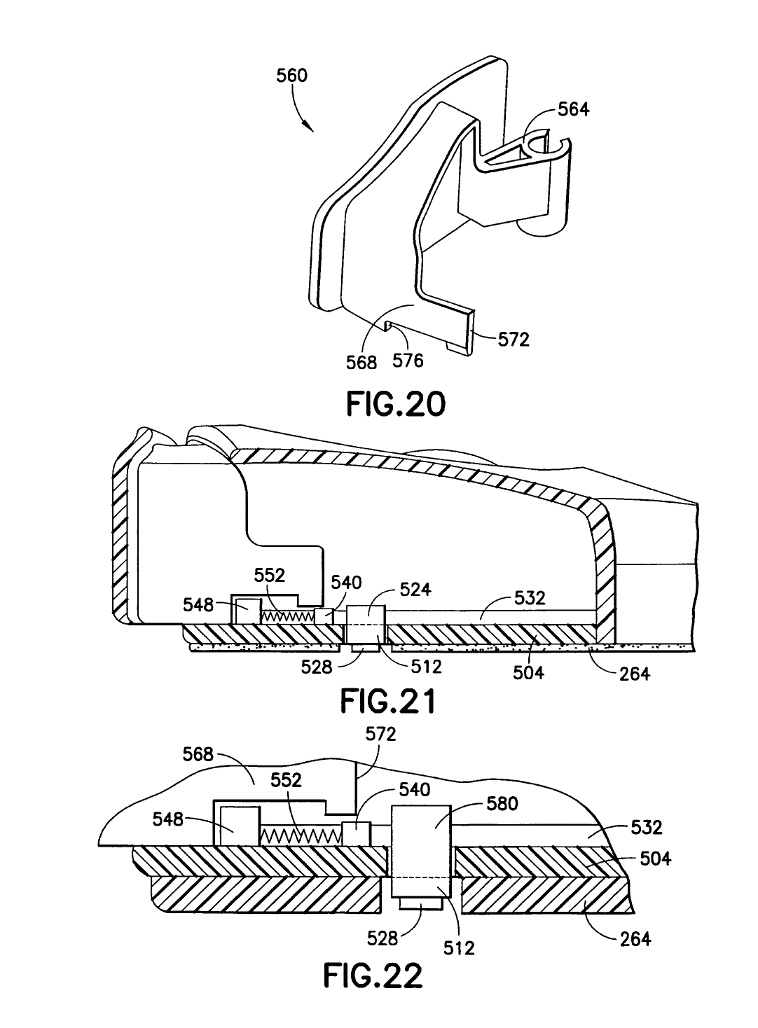

FIG. 20 illustrates an embodiment of the activator button 560. Similar to the activator button 128 described above, the activator button has a hinge arm 564 and an activation arm 568. The hinge arm 224 of the activator button 560 includes a cylindrical portion with an opening for selective connection and rotation about first post 192 (see, for example, FIG. 4) of the body 504. The activation arm 568 includes a rotor-bearing surface 572 for contacting and rotating the rotor 136 upon activation of the infusion device 500. The activation arm 568 also has a cutout on a lower portion thereof, including the biasing-bearing surface 576 for contacting and moving the biasing member 548 upon activation of the infusion device 500. Though the rotor 136 is not illustrated in FIGS. 17-27, according to one embodiment, the safety member 532 is disposed beneath the rotor 136 and moves independently thereof.

According to one embodiment, in a pre-deployed position, shown, for example, in FIG. 21, the external surface (i.e., a patient surface) of the blocker 512 is substantially flush with a patient-surface of the body 504. Additionally, the blocker adhesive 528 is substantially flush with the adhesive pad 264 disposed on the patient-surface of the body 504. The blocker 512 is sized to provide a friction fit with the blocker opening 520. More specifically, a latitudinal width of the blocker 512 is sized to provide a friction fit with the blocker opening 520, to maintain the blocker 512 in the pre-deployed position prior to deployment.

In a pre-deployed position of another embodiment of blocker 512, shown, for example, in FIG. 22, the external surface (i.e., a patient surface) of the blocker 512 is adjacent to and substantially parallel with a patient-surface of the body 504. In this embodiment, the blocker post 580 is sized to provide a friction fit with the blocker opening 520. More specifically, a latitudinal width of the blocker post 580 is sized to provide a friction fit with the blocker opening 520 to maintain the blocker 512 in the pre-deployed position prior to deployment. In this embodiment, because the blocker post 580 is taller than the embodiment incorporating blocker post 524, to ensure that the rotor bearing surface of the activation arm 568 is able to pass over the blocker post 580 when the activator button 560 is activated by the patient, it may be necessary to reshape the distal end of the activation arm 568, as shown, for example, in FIG. 22.

According to one embodiment, the blocker adhesive 528 is stronger than the body adhesive 264, to ensure rotation of the blocker 512 away from the body 504 during removal of the body 504 from the patient. In other words, because the blocker adhesive 528 is stronger than the body adhesive 264, the body adhesive 264 releases from the patient's skin before the blocker adhesive 528 releases, and thus, the blocker 512 remains adhered to the patient's skin at least long enough to rotate the blocker 512 away from the body 504. According to another embodiment, the blocker adhesive 528 may be the same strength as the body adhesive 264, or even less strong than the body adhesive, so long as when the patient removes the infusion device 500, the interaction between the blocker adhesive 528 and the patient's skin overcomes the friction fit between the blocker 512 and the blocker opening 520, to rotate the blocker 512 away from the body 504.

For clarity, FIG. 25 illustrates a more fully exploded view of the infusion device 500 in the pre-activated state. As shown in FIG. 25, the blocking tab 540 is not yet biased toward the blocker post 524, and thus, the safety member 532 is not yet biased toward the needle opening 508. Additionally, because the activator button 560 is not yet been activated, the microneedles 152 (fluidly connected to the channel 172 via needle manifold 584), remain inside the body 504.

In operation, subsequent to the adherence of the infusion device 500 to the patient's skin, when the patient activates the infusion device 500 by depressing the activator button 560, the movement of the activator button 560 opens the valve 168, establishing a fluid communication path between the reservoir 160 and the microneedles 152. Additionally, with the activation of the infusion device 500, the rotor bearing surface 572 of the activator button 560 contacts and rotates the rotor 136 about the cylindrical housing 200, thereby releasing plunger 144 and the pressurization spring 140 and allowing a plunger 144 to press against the flexible film (reservoir dome seal 164) of the reservoir 160, thereby pressurizing the reservoir. Further, the activation of the infusion device 500 releases the drive spring 148 from the drive spring holder 260 of the rotor 136, thereby driving the microneedles 152 to extend outside the infusion device 500 (through the needle opening 508 in the body 504) and seat the microneedles 152 within the patient.

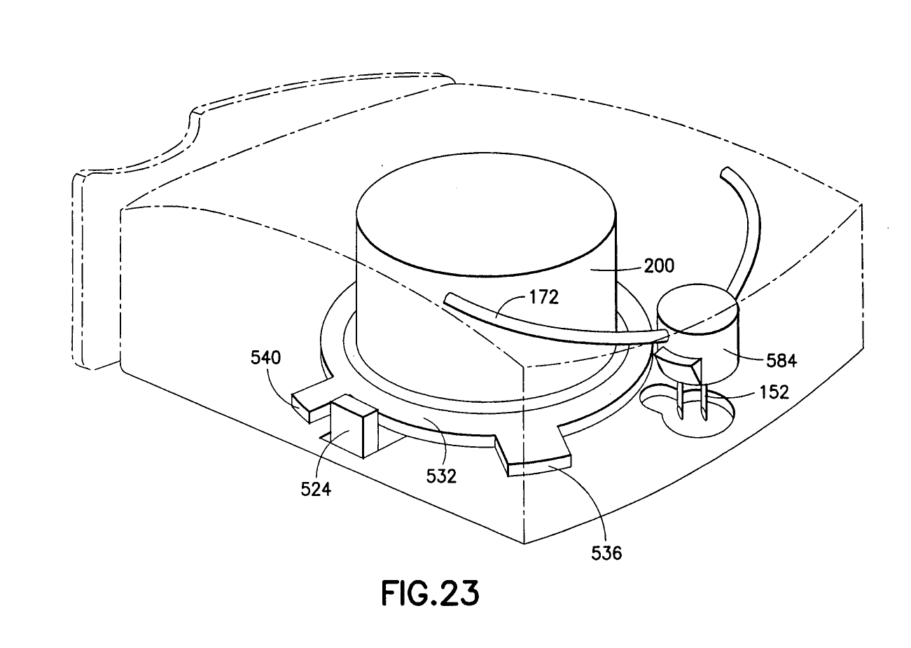

Furthermore, during the movement of the activator button 560 to the activated position, the biasing bearing surface 576 contacts and moves the biasing member 548, whose movement compresses the biasing spring against the blocking tab 540 of the safety member 532, to bias the safety member 532 toward the needle opening 508. Similarly, in the embodiment employing the safety bearing member 556, the biasing bearing surface 576 contacts and moves the biasing member 548, whose movement compresses the biasing spring against the safety bearing member 556, which moves in response and contacts the blocking tab 540 of the safety member 532, to bias the safety member 532 toward the needle opening 508. As a result, in the pre-deployed position shown, for example, in FIGS. 23 and 24, the blocking tab 540 of the safety member 532 contacts the blocker 512, which prevents movement of the safety member 532 toward the needle opening 508.

Once delivery of the medicament is complete, the patient begins removing the infusion device 500 from the patient's skin. As previously noted, the interaction between the blocker adhesive 528 and the patient's skin is sufficient to overcome the friction fit between the blocker 512 and the blocker opening 520 to rotate the blocker 512 away from the body 504. Then, because the blocker 512 rotates and no longer contacts the blocking tab 540 of the safety member 532, the covering tab 536 rotates toward the needle opening 508 under the force of the biasing spring 552.

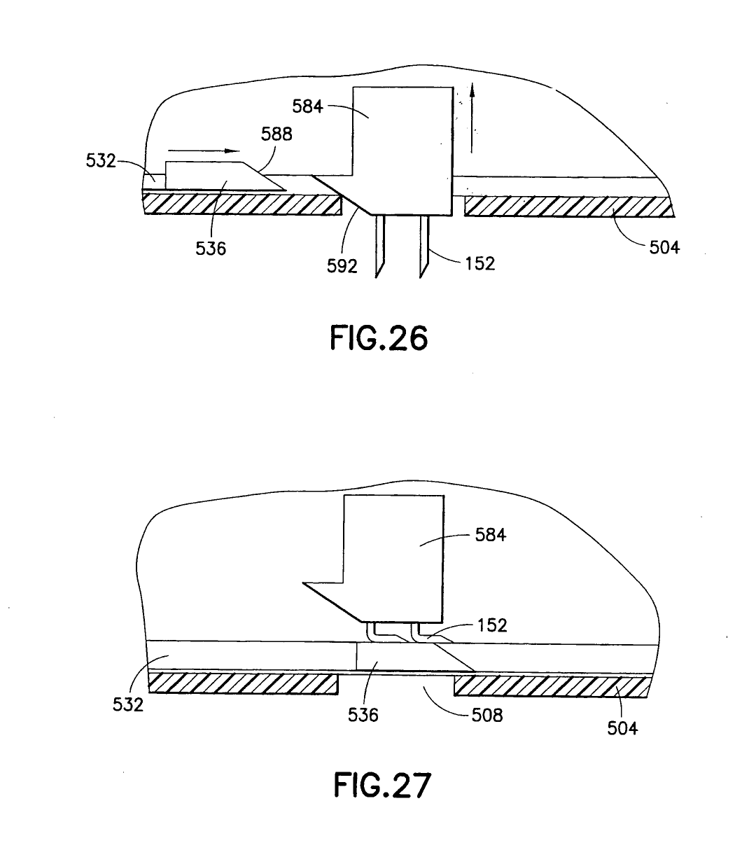

FIG. 26 illustrates the interaction between the safety member 532 and the needle manifold 584 when the safety mechanism of the infusion device 500 is deployed. As the safety member or covering ring 532 moves to a covered position covering the needle opening 508, the safety member 532 retracts the needle manifold 584 into the body 504 and bends the microneedles 152. More specifically, according to one embodiment, as shown in FIG. 26, the covering tab 536 has a beveled leading edge or ramp 588 and the needle manifold 584 has a corresponding beveled edge or ramp 592. Due to the force of the biasing spring 552, as the covering tab 536 rotates to the covered position, the beveled leading 588 of the covering tab 536 engages the manifold ramp 592, thereby driving the needle manifold 584 upward and bending the microneedles 152, to retract the manifold 584 and microneedles 152 into the body 504 and cover the needle opening 508, as shown in FIG. 27.

Thus, as shown in FIGS. 17-27, the infusion device 500 includes a safety mechanism that automatically retracts the microneedles 152 into the body 504 and covers the needle opening 508 upon removal of the infusion device 500 from the patient's skin.

It will be understood that portions of the infusion device 500 not expressly disclosed are substantially similar to the corresponding portions of the infusion device 100.

With respect to the safety mechanism of the infusion device 100, one advantage of the safety mechanism of the infusion device 500 is that the pivoting housing/shield (safety mechanism 108) is not required, thereby providing for a more compact infusion device. Nevertheless, though not illustrated, it is possible to combine features of the disclosed safety mechanisms. For example, the safety mechanism 108 could employ a rotating plate that covers the needle opening 156 subsequent to the deployment of safety mechanism 108.

Although only a few exemplary embodiments of the present invention have been described in detail above, those skilled in the art will readily appreciate that many modifications are possible in the exemplary embodiments without materially departing from the novel teachings and advantages of this invention. Accordingly, all such modifications are intended to be included within the scope of the appended claims and equivalents thereof.

1. A drug delivery device, comprising:
a body having a surface placeable on the skin of a patient, the body having a needle opening and a reservoir disposed therein for containing a medicament;
an injection needle for penetrating the skin of a patient, the needle providing a path for the medicament between the reservoir and the patient, and being selectively movable between a first position, in which a part of the needle is contained within the body, and a second position, in which a part of the needle protrudes from the body through the needle opening; and
safety means for automatically moving the needle from the second position to the first position, and for covering the needle opening upon removal of the device from the skin of the patient.

2. The device according to claim 1, wherein:
the body has a blocker opening; and
the safety means comprises:
a safety member;
a biasing mechanism biasing the safety member toward the needle opening upon activation of the device; and
a blocker movably connected to the body for selectively preventing movement of the safety member toward the needle opening prior to deployment of the safety means;
wherein movement of the safety member to a covered position covering the needle opening retracts the needle within the body.

3. The device according to claim 2, wherein the blocker includes a blocker post extending therefrom for selectively engaging the safety member to prevent movement of the safety member toward the needle opening prior to deployment of the safety means.

4. The device according to claim 3, wherein:
the safety member comprises:
a covering tab extending therefrom for covering the needle opening; and
a blocking tab extending therefrom;
wherein the blocker post engages the blocking tab to prevent movement of the safety member toward the needle opening prior to deployment of the safety means.

5. The device according to claim 4, wherein:
the blocker is hingeably connected with the body;
an external surface of the blocker comprises a blocker adhesive, such that upon removal of the body from the patient, interaction between the blocker adhesive and the patient rotates the blocker away from the body, thereby disengaging the blocker post from the blocking tab and permitting the safety member to move toward the needle opening.

6. The device according to claim 5, wherein:
the external surface of the blocker is substantially flush with a patient-surface of the body in a pre-deployed position of the blocker; and
the blocker is sized to provide a friction fit with the blocker opening to maintain the blocker in the pre-deployed position prior to deployment of the safety means.

7. The device according to claim 5, wherein:
the external surface of the blocker is adjacent to and substantially parallel with the surface of the body in a pre-deployed position of the blocker; and
the blocker post is sized to provide a friction fit with the blocker opening to maintain the blocker in the pre-deployed position prior to deployment of the safety means.

8. The device according to claim 5, wherein:
the device further comprises a body adhesive disposed on the surface of the body for adhering the body to the patient during use; and
the blocker adhesive is stronger than the body adhesive, to ensure rotation of the blocker away from the body during removal of the body from the patient.

9. The device according to claim 4, wherein:
the device further comprises an activator button for activating the device;
the biasing mechanism comprises a biasing member and a biasing spring connected thereto; and
the biasing mechanism is disposed such that upon activation of the device by pressing of the activator button, the activator button contacts the biasing member, which in turn compresses the biasing spring against the blocking tab to bias the safety member toward the needle opening.

10. The device according to claim 4, wherein:
the body comprises a substantially cylindrical housing in which the reservoir is disposed; and
the safety member comprises a covering ring rotatably disposed about the cylindrical housing.

11. The device according to claim 10, wherein rotation of the covering ring to the covered position bends the needle, thereby retracting the needle within the body.

12. The device according to claim 10, wherein:
the device further comprises a needle manifold connected to the needle and disposed on the path between the reservoir and the needle; and
the covering ring comprises a ramp such that during rotation of the covering ring to the covered position, the ramp contacts the needle manifold, thereby retracting the needle within the body.

13. A safety mechanism for a drug delivery device including a body having a surface placeable on the skin of a patient and a needle opening, a reservoir disposed in the body for containing a medicament, and an injection needle for penetrating the skin of a patient, the needle providing a path for the medicament between the reservoir and the patient, and being selectively movable between a first position, in which a part of the needle is contained within the body, and a second position, in which a part of the needle protrudes from the body through the needle opening, the safety mechanism comprising:
a safety member movable within the body from a pre-deployed position to a covered position covering the needle opening;
a biasing mechanism biasing the safety member toward the covered position upon activation of the drug delivery device; and
a blocker movably connected to the body for selectively preventing movement of the safety member toward the covered position prior to deployment of the safety mechanism;
wherein movement of the safety member to the covered position retracts the needle within the body.

14. The safety mechanism according to claim 13, wherein:
the biasing mechanism comprises a biasing member and a biasing spring connected thereto; and
the biasing mechanism is disposed such that upon activation of the drug delivery device, the biasing member compresses the biasing spring against the blocking tab to bias the safety member toward the covered position.

15. The safety mechanism according to claim 14, wherein the blocker includes a blocker post extending therefrom for selectively engaging the safety member to prevent movement of the safety member toward the covered position prior to deployment of the safety mechanism.

16. The safety mechanism according to claim 15, wherein:
the safety member comprises:
a covering tab extending therefrom for covering the needle opening; and
a blocking tab extending therefrom;
wherein the blocker post engages the blocking tab prior to deployment of the safety means to prevent movement of the safety member toward the covered position.

17. The safety mechanism according to claim 16, wherein:
the blocker is hingeably connected with the body;
an external surface of the blocker comprises an adhesive, such that upon removal of the drug delivery device from the patient, interaction between the adhesive and the patient rotates the blocker away from the body, thereby disengaging the blocker post from the blocking tab and permitting the safety member to move toward the covered position.

18. The safety mechanism according to claim 17, wherein:
the external surface of the blocker is substantially flush with a patient-surface of the body in a pre-deployed position of the blocker; and
the blocker is sized to provide a friction fit with a blocker opening of the body to maintain the blocker in the pre-deployed position prior to deployment of the safety mechanism.

19. The safety mechanism according to claim 17, wherein:
the external surface of the blocker is adjacent to and substantially parallel with the surface of the body in a pre-deployed position of the blocker; and
the blocker post is sized to provide a friction fit with a blocker opening of the body to maintain the blocker in the pre-deployed position prior to deployment of the safety mechanism.

20. The safety mechanism according to claim 16, wherein:
the body comprises a substantially cylindrical housing in which the reservoir is disposed; and
the safety member comprises a covering ring rotatably disposed about the cylindrical housing.

21. The safety mechanism according to claim 20, wherein rotation of the covering ring to the covered position bends the needle, thereby retracting the needle within the body.

22. The safety mechanism according to claim 20, wherein:
the drug delivery device further comprises a needle manifold connected to the needle and disposed on the path between the reservoir and the needle; and
the covering ring comprises a ramp such that during rotation of the covering ring to the covered position, the ramp contacts the needle manifold, thereby retracting the needle within the body.

23. A drug delivery device, comprising:
a body having a surface placeable on the skin of a patient, the body having a needle opening and a reservoir disposed therein for containing a medicament;
an injection needle for penetrating the skin of a patient, the needle providing a path for the medicament between the reservoir and the patient, and being selectively movable between a first position, in which at least a part of the needle is contained within the body, and a second position, in which at least a part of the needle protrudes from the body through the needle opening; and
a safety mechanism pivotably connected to the body and being selectively pivotable between a retracted position and a deployed position shielding the injection needle, the safety mechanism having a safety surface placeable on the skin of the patient, the safety surface having an adhesive disposed thereon for adhering the safety mechanism to the skin of the patient and thereby automatically pivoting the safety mechanism to the second position upon removal of the drug delivery device from the skin of the patient.

24. The device according to claim 23, wherein the safety mechanism is biased toward the retracted position.

25. The device according to claim 24, wherein the safety mechanism comprises a pair of locking posts disposed to contact opposing edges of the needle opening and bias the safety mechanism toward the first position.

26. The device according to claim 25, wherein each locking post comprises:
a post extending portion extending substantially perpendicularly from an inner surface of the safety mechanism; and
a wedge portion disposed at a distal end of the post extending portion;
wherein as a height of the wedge portion increases with respect to the inner surface of the safety mechanism, a width of the wedge portion increases.

27. The device according to claim 26, wherein:
as the safety mechanism pivots from the retracted position to the deployed position, the wedge portions act against the respective opposing edges of the needle opening causing the locking posts to deform elastically toward one another; and
as the safety mechanism reaches the deployed position, top edges of the wedge portions pass bottom edges of the needle opening and the locking posts snap back to their substantially un-deformed states and contact an external surface of the body to maintain the safety mechanism in the second position.

28. The device according to claim 23, wherein the safety mechanism comprises:
a rim portion defining an external surface of the safety mechanism; and
a shield extending above the rim portion for shielding the injection needle when the safety mechanism is deployed in the deployed position.

29. The device according to claim 23, wherein the device comprises a rotor disposed in the body and being pivotable from a pre-activated position for selectively maintaining the injection needle in the first position to an activated position, the rotor having a safety retaining projection; and
wherein the safety mechanism comprises a guide post extending through the body and engaging the safety retaining projection when the safety mechanism is in the retracted position, for preventing movement of the safety mechanism prior to movement of the injection needle to the second position.
